(19) United States
(12) Patent Application Publication
(10) Pub. No.: US 2023/0028700 A1
(43) Pub. Date: Jan. 26, 2023

(54) CHIRAL GUANIDINES, SALTS THEREOF, METHODS OF MAKING CHIRAL GUANIDINES AND SALTS THEREOF, AND USES OF CHIRAL GUANIDINES AND SALTS THEREOF IN THE PREPARATION OF ENANTIOMERICALLY PURE AMINO ACIDS

(71) Applicants: Rui FU, Toronto (CA); Jik CHIN, Toronto (CA); Soon Mog SO, Toronto (CA)

(72) Inventors: Rui FU, Toronto (CA); Jik CHIN, Toronto (CA); Soon Mog SO, Seoul (KR)

(21) Appl. No.: 17/773,561

(22) PCT Filed: Nov. 2, 2020
(86) PCT No.: PCT/CA2020/051488

§ 371 Date: Apr. 29, 2022

(30) Foreign Application Priority Data
Oct. 31, 2019 (GB) 1915826.0

(51) Int. Cl. C07D 233/46; C07D 403/12

(52) U.S. Cl. CPC C07D 233/46; C07D 403/12; C07B 2200/07

(57) ABSTRACT

Provided are compounds and salts having a structure of Formula (I) or (II): (I), and (II) wherein: both of the chiral carbon atoms denoted by “*” are both in the R configuration or both in the S configuration. Compounds and salts of Formulae (I) and (II) are useful in the preparation of enantiomerically pure amino acids. Conversion of amino acids to D-form from any of L-form, racemate or other enantiomerically impure mixtures or conversion of amino acids to L-form from any of D-form, racemate or other enantiomerically impure mixtures is disclosed.

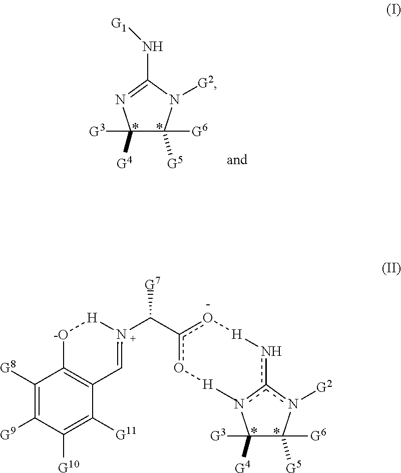

### TECHNICAL FIELD

This invention relates to the field of chemistry and in particular to chiral guanidines, methods of making them and their use in the preparation of enantiomerically pure amino acids.

### BACKGROUND

D-amino acids are common building blocks to many pharmaceuticals such as saxagliptin (antidiabetic), tadalafil (erectile dysfunction), clopidogrel (heart disease) and cycloserine (antibiotic). In addition, D-amino acids are of considerable interest in the field of unnatural peptide-based drugs such as telaprevir (antiviral), degarelix (prostate cancer), and carfilzomib (multiple myeloma). Many chemical and biological methods have been developed for making D-amino acids. Chemical processes include Strecker synthesis, hydrogenation, transamination, alkylation, arylation, and alcoholysis reactions.

Stereoselective organic and cobalt-based receptors for L to D conversion of alpha-amino acids have also been reported as well as highly stereoselective nickel-based receptors for deracemisation of a wide variety of natural and unnatural alpha-amino acids.

So, S. M., et al., “Highly Stereoselective Recognition and Deracemization of Amino Acids by Supramolecular Self-Assembly”, Angew. Chem. Int. Ed. (2014), 53 (3), 829-832 describe the highly stereoselective supramolecular self-assembly of alpha-amino acids with a chiral aldehyde derived from binol and a chiral guanidine derived from diphenylethylenediamine (dpen) to form the imino acid salt. This system can be used to cleanly convert D-amino acids into L-amino acids or vice versa at ambient temperature. It can also be used to synthesize alpha-deuterated D- or L-amino acids. A crystal structure of the ternary complex together with DFT computation provided detailed insight into the origin of the stereoselective recognition of amino acids.

Moozeh, K., et al., “Catalytic Stereoinversion of L-Alanine to Deuterated D-Alanine”, Agnew. Chem. Int. Ed. (2015), 54 (32), 9381-9385 describe a combination of an achiral pyridoxal analogue and a chiral base developed for catalytic deuteration of L-alanine with inversion of stereochemistry to give deuterated D-alanine under mild conditions (neutral pD and 25° C.) without the use of any protecting groups. This system can also be used for catalytic deuteration of D-alanine with retention of stereochemistry to give deuterated D-alanine. Thus a racemic mixture of alanine can be catalytically deuterated to give an enantiomeric excess of deuterated D-alanine. While catalytic deracemization of alanine is forbidden by the second law of thermodynamics, this system can be used for catalytic deracemization of alanine with deuteration. Such green and biomimetic approach to catalytic stereocontrol provides insights into efficient amino acid transformations.

### SUMMARY

This invention is based, at least in part, on the elucidation of chemical molecules suitable for use in processes for making stereospecific amino acid molecules. Also provided are methods and processes for making stereospecific amino acids. Solubility-induced diastereomeric transformation (SIDT) for deracemisation of amino acids is provided herein. This process may provide increased yield of classical resolutions and does not require the development of stereoselective receptors. SIDT relies on small solubility differences between equilibrating diastereomers. The term SIDT is not to be confused with the term “CIDT” (Crystalization-induced diastereomeric transformation) or “CIET” (crystallization-induced enantiomeric transformation) because crystallization is not required for diastereomer transformation as may be for enantiomer transformation where conglomerate crystals may be required. Herein the term SIDT is used as crystallization is not always necessary for processes described herein because processes described herein often rely on small solubility differences between equilibrating diastereomers. Although CIDT has been previously used for deracemisation of amino esters and amino amides, it has been a challenge to deracemise free unactivated amino acids due to the high pKₐ of the alpha-proton. Using two types of strong hydrogen bonds in concert the present invention enables deracemisation of unactivated amino acids using SIDT under mild reaction conditions (See FIG. 1). The same method can be used to convert readily available L amino acids to D amino acids and vice-versa as well as the deracemisation of racemic (or other enantiomerically impure mixtures) natural and unnatural amino acids.

In illustrative embodiments of the present invention, there is provided a compound having a structure of Formulae (I), (Ia) and/or (Ib):

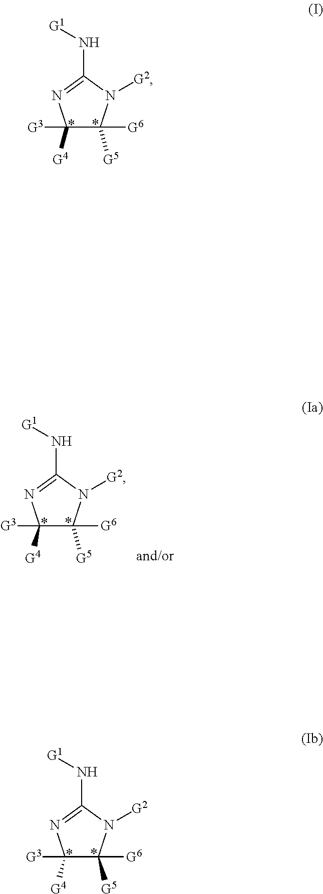

wherein:

both of the chiral carbon atoms denoted by “*” are both in the R configuration or both in the S configuration;

G¹ is selected from the group consisting of: H, C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)];

G² is selected from the group consisting of: H, C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)];

G³ and G⁶ are independently selected from the group consisting of: H, C₁-C₁₂ substituted alkyl, C₂-C₁₂ unsubstituted alkyl, C₁-C₁₂ substituted heteroalkyl, and C₁-C₁₂ unsubstituted heteroalkyl;

G⁴ and G⁵ are independently selected from the group consisting of: unsubstituted C₆-C₁₂ aryl, substituted C₆-C₁₂ aryl, unsubstituted C₆-C₁₂ heteroaryl, substituted C₆-C₁₂ heteroaryl, unsubstituted [(C₆ aryl)-(C₁-C₆ alkyl)-(C₆ aryl)], substituted [(C₆ aryl)-(C₁-C₆ alkyl)-(C₆ aryl)], unsubstituted [(C₆ aryl)-(C₁-C₆ heteroalkyl)-(C₆ aryl)], and substituted [(C₆ aryl)-(C₁-C₆ heteroalkyl)-(C₆ aryl)];
provided that:

(i) either:
(a) at least one of G¹, G², G³, and G⁶ is not H; or
(b) at least one of G⁴ and G⁵ is not phenyl;

(ii) if G⁴ and G⁵ are both

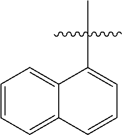 [SMILES: *c1cccc2ccccc12]

or both

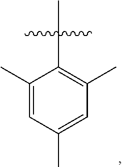

then at least one of G¹, G², G³ and G⁶ is not H;

(iii) if G³ and G⁶ are both H, and G² is methyl,

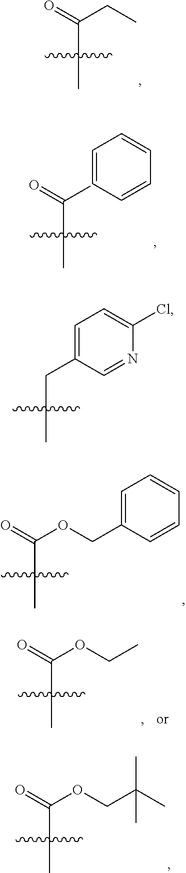

then either G⁴ and G⁵ are not both phenyl, or G¹ is not NO₂, ethyl, tert-butyl, benzyl, cyclohexanyl,

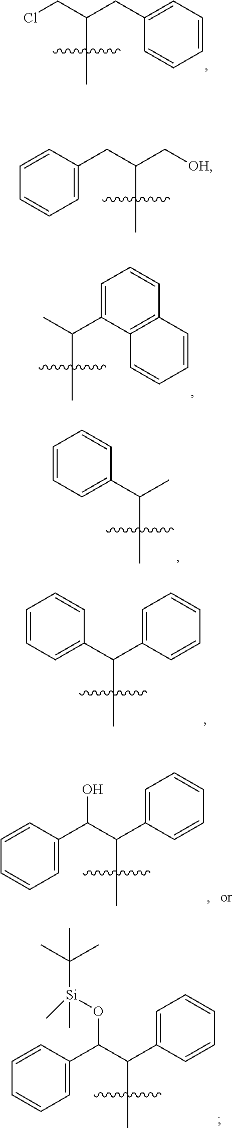

(iv) if G², G³, and G⁶ are all H, and G⁴ and G⁵ are both phenyl, then G¹ is not H, methyl, ethyl, tert-butyl, phenyl, benzyl, cyclohexanyl,

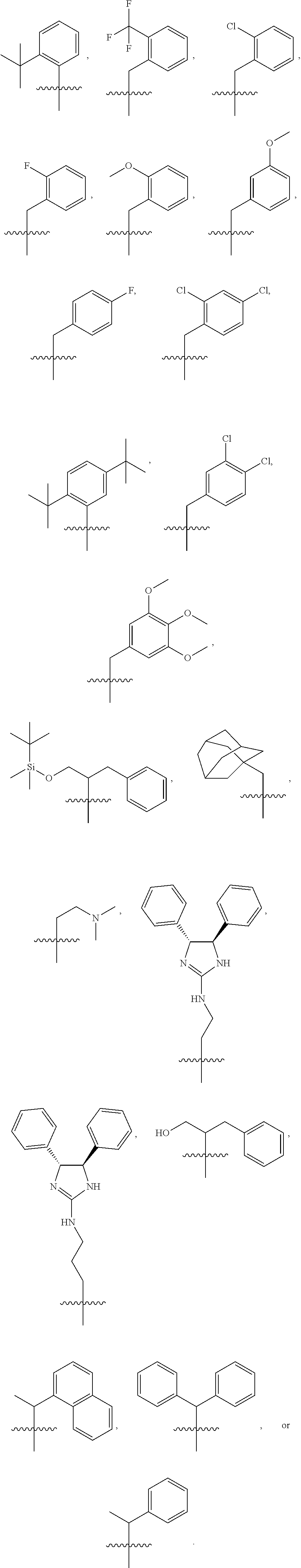

In illustrative embodiments of the present invention, there is provided a compound described herein wherein if G⁴ and G⁵ are both

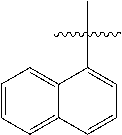 [SMILES: *c1cccc2ccccc12]

or both

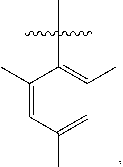 [SMILES: *C(=C\C)/C(C)=C\C(=C)C]

then G¹ is not NO₂, phenyl,

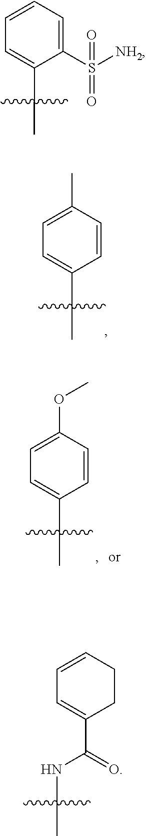

In illustrative embodiments of the present invention, there is provided a compound described herein wherein if G³ and G⁶ are both H, and G² is methyl,

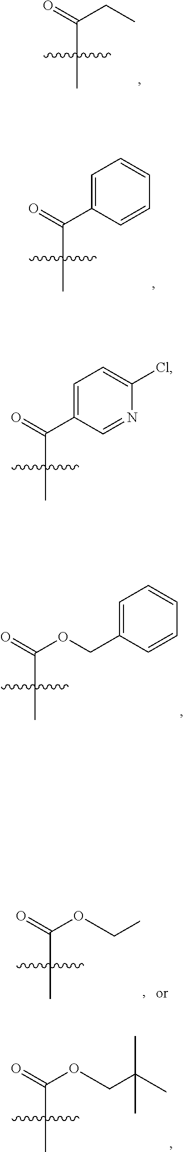

then G¹ is not H.

In illustrative embodiments of the present invention, there is provided a compound described herein wherein if G², G³, and G⁶ are all H, and G⁴ and G⁵ are both phenyl, then G¹ is not NO₂,

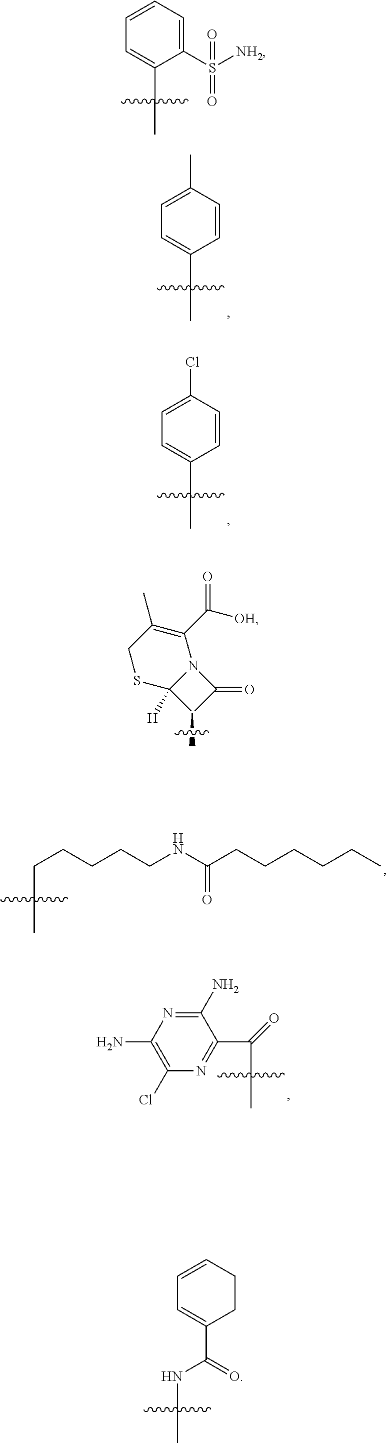

In illustrative embodiments of the present invention, there is provided a compound described herein wherein the G¹ is not benzyl, substituted benzyl, benzoyl, substituted benzoyl, or —CH₂-cyclohexanyl.

In illustrative embodiments of the present invention, there is provided a compound described herein wherein the both of the chiral carbon atoms denoted by “*” are both in the S configuration.

In illustrative embodiments of the present invention, there is provided a compound described herein wherein the both of the chiral carbon atoms denoted by “*” are both in the R configuration.

In illustrative embodiments of the present invention, there is provided a compound described herein wherein G¹ is selected from the group consisting of: H, C₁-C₆ unsubstituted alkyl, C₁-C₆ substituted alkyl, C₆ unsubstituted aryl, C₆ substituted aryl, unsubstituted (C₁-C₆ alkylene)-(C₆ aryl), substituted (C₁-C₆ alkylene)-(C₆ aryl), C₁-C₆ unsubstituted heteroalkyl, C₁-C₆ substituted heteroalkyl, C₆ unsubstituted heteroaryl, C₆ substituted heteroaryl, unsubstituted hetero[(C₁-C₆ alkylene)-(C₆ aryl)], and substituted hetero[(C₁-C₆ alkylene)-(C₆ aryl)].

In illustrative embodiments of the present invention, there is provided a compound described herein wherein G² is selected from the group consisting of: H, C₁-C₆ unsubstituted alkyl, C₁-C₆ substituted alkyl, C₆ unsubstituted aryl, C₆ substituted aryl, unsubstituted (C₁-C₆ alkylene)-(C₆ aryl), substituted (C₁-C₆ alkylene)-(C₆ aryl), C₁-C₆ unsubstituted heteroalkyl, C₁-C₆ substituted heteroalkyl, C₆ unsubstituted heteroaryl, C₆ substituted heteroaryl, unsubstituted hetero[(C₁-C₆ alkylene)-(C₆ aryl)], and substituted hetero[(C₁-C₆ alkylene)-(C₆ aryl)].

In illustrative embodiments of the present invention, there is provided a compound described herein wherein G² is selected from the group consisting of: H, C₁-C₁₂ unsubstituted alkyl, C₁-C₁₂ substituted alkyl, C₆-C₁₂ unsubstituted aryl, C₆-C₁₂ substituted aryl, C₁-C₁₂ unsubstituted heteroalkyl, C₁-C₁₂ substituted heteroalkyl, C₆-C₁₂ unsubstituted heteroaryl, C₆-C₁₂ substituted heteroaryl.

In illustrative embodiments of the present invention, there is provided a compound described herein wherein G² is selected from the group consisting of: H, C₁-C₆ unsubstituted alkyl, C₁-C₆ substituted alkyl, C₆-C₈ unsubstituted aryl, C₆-C₈ substituted aryl, C₁-C₆ unsubstituted heteroalkyl, C₁-C₆ substituted heteroalkyl, C₅-C₈ unsubstituted heteroaryl, C₅-C₈ substituted heteroaryl.

In illustrative embodiments of the present invention, there is provided a compound described herein wherein G³ and G⁶ are independently selected from the group consisting of: H, C₁-C₆ substituted alkyl, C₂-C₆ unsubstituted alkyl, C₁-C₆ substituted heteroalkyl, and C₁-C₆ unsubstituted heteroalkyl.

In illustrative embodiments of the present invention, there is provided a compound described herein wherein G⁴ and G⁵ are independently selected from the group consisting of: unsubstituted C₆ aryl, substituted C₆ aryl, unsubstituted C₆ heteroaryl, and substituted C₆ heteroaryl.

In illustrative embodiments of the present invention, there is provided a compound described herein wherein the compound has no acid groups.

In illustrative embodiments of the present invention, there is provided a compound described herein wherein the compound has no charged groups.

In illustrative embodiments of the present invention, there is provided a compound described herein wherein G³ and G⁶ are both H.

In illustrative embodiments of the present invention, there is provided a compound described herein wherein G² is H.

In illustrative embodiments of the present invention, there is provided a compound described herein wherein G⁴ and G⁵ are both a substituted phenyl.

In illustrative embodiments of the present invention, there is provided a compound described herein wherein G⁴ and G⁵ are phenyl.

In illustrative embodiments of the present invention, there is provided a compound described herein wherein G¹ is H.

In illustrative embodiments of the present invention, there is provided a compound selected from the group consisting of:

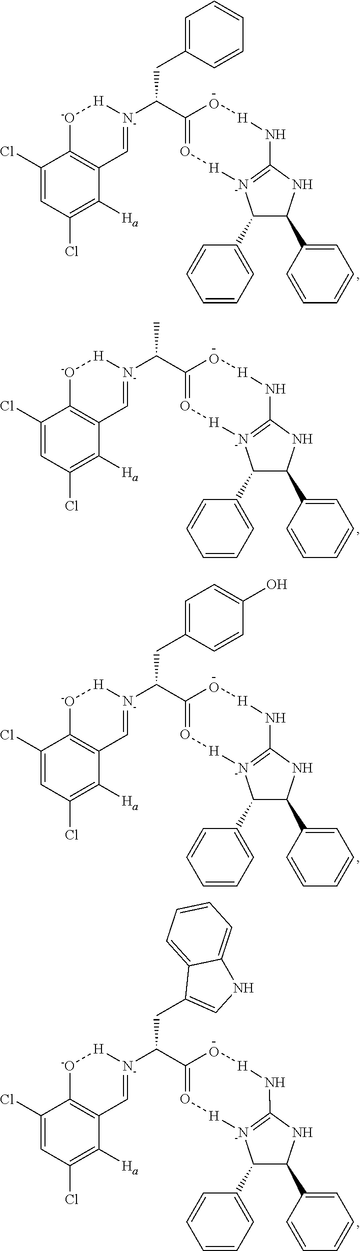

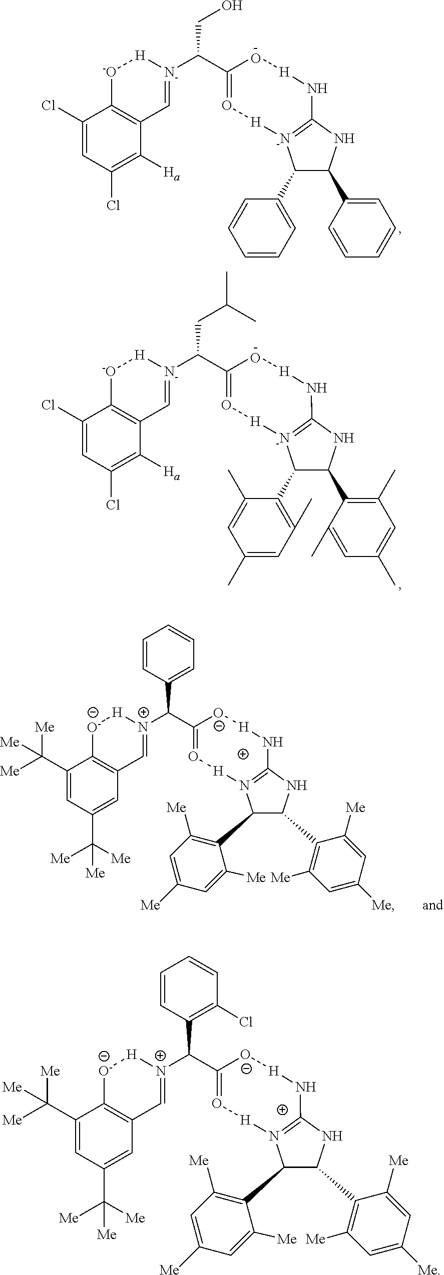

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

FIG. 1 illustrates substrates for solubility-induced diastereomeric transformation (SIDT) strategy of L to D conversion. In FIG. 1, Mes=1,3,5-trimethylbenzene.

FIG. 2. a) Partial ¹H NMR (CDCl₃) of a mixture of phenylalanine imine salt complexes (prior to SIDT) showing Hₐ belonging to (S,S)-D-6a and (S,S)-L-6a. The alpha-proton and methylene protons of phenylalanine are shown on the right. b) Partial ¹H NMR (CDCl₃) of the phenylalanine imine salt complex after SIDT.

FIG. 3 illustrates the X-ray crystal structure of (S,S)-D-6d.

FIG. 4. a) Proposed concerted general acid/base catalysis for racemisation. b) Computed structure of an anionic glycine imino acid salt complex with an alpha-proton deprotonated. (B3LYP/6-31 G* level of theory)

FIG. 5: Illustrates imine salt complexes of Tryptophan together with ¹H NMR data. The top partial ¹H NMR (CDCl₃) of the tryptophan imine salt complexes (prior to SIDT) shows the ortho 3,5-dichlorosalicylaldehyde protons belonging to the heterochiral complex (S,S-D), the homochiral complex (S,S-L). The alpha-proton and methylene protons of tryptophan are shown on the right. The bottom partial ¹H NMR (CDCl₃) of the tryptophan imine salt complex is illustrative of the complexes after undergoing SIDT. D:L ratio of imine salt complex as determined by ¹H NMR is 98:2.

FIG. 6: Illustrates imine salt complexes of 2-chlorophenylglycine together with ¹H NMR data. The top partial ¹H NMR (CDCl₃) of the 2-chlorophenylglycine imine salt complexes (prior to SIDT) shows the para 3-tert-butylsalicylaldehyde protons belonging to the heterochiral complex (R,R-L), and the homochiral complex (R,R-D). The imine protons of the diastereomeric salts are shown downfield. The bottom partial ¹H NMR (CDCl₃) of the 2-chlorophenylgycine imine salt complex is illustrative of the complexes after undergoing SIDT. L:D ratio of imine salt complex as determined by ¹H NMR is 97:3.

### DETAILED DESCRIPTION

As used herein, the term “alkyl,” by itself or as part of another substituent, means, unless otherwise stated, a straight chain or branched chain, or cyclic hydrocarbon radical, or combinations thereof, which may be fully saturated, mono-unsaturated or polyunsaturated, having the number of carbon atoms designated (i.e. C₁-C₁₀ or 1- to 10-membered means having one to ten carbons).

Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

As used herein, the term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, with those groups having 12 or fewer carbon atoms being preferred in the present invention. In some embodiments, alkyl groups may have from 2 to 12 carbon atoms and in some embodiments alkyl group may have from 1 to 6 carbon atoms.

As used herein, the term “heteroalkyl,” by itself or in combination with another term, means, unless otherwise stated, a straight chain or branched chain, or cyclic monovalent radical, or combinations thereof, consisting of the stated number of carbon atoms and at least one heteroatom selected from the group consisting of O, N, Si and S, and wherein the nitrogen and sulfur atoms may optionally be oxidized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—C(═O)—CH₃, —CH₂—CH₂—CH₂—C(═O)—O—C(CH₃)—CH₃, —CH₂—CH₂—CH₂—C(═O)—N—CH(CH₃), —CH₂—CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, unless otherwise clear from context, no orientation of the linking group is implied by the direction in which the formula of the linking group is written. For example, the formula —C(O)₂R′— represents both —C(O)₂R′— and —R′C(O)₂—.

As used herein, the terms “cycloalkyl” and “heterocycloalkyl”, by themselves or in combination with other terms, represent, unless otherwise stated, cyclic versions of “alkyl” and “heteroalkyl”, respectively. Thus, a cycloalkyl or heterocycloalkyl include saturated and unsaturated ring linkages.

Additionally, for heterocycloalkyl, a heteroatom can occupy the position at which the heterocycle is attached to the remainder of the molecule or in an interior position of the heterocycloalkyl group. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

As used herein, the terms “halo” or “halogen,” by themselves or as part of another substituent, mean, unless otherwise stated, a fluorine, chlorine, bromine, or iodine atom or radical. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

As used herein, the term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized. A heteroaryl group can be attached to the remainder of the molecule through a heteroatom or a carbon atom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridinyl, 3-pyridinyl, 4-pyridinyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described herein.

As used herein, the term “substituted” refers to the replacement of a hydrogen atom on a compound with a substituent and/or substituent group. A substituent may be a non-hydrogen atom or multiple atoms of which at least one is a non-hydrogen atom and one or more may or may not be hydrogen atoms. For example, without limitation, substituted compounds may comprise one or more substituents selected from the group consisting of: —OR′, ═O, ═NR′, ═N—OR′, —NR′R″, —SR′, -halogen, —SiR′R″ R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR′—C(NR″R″′)═NR″″, —S(O)R′, —S(O)₂R′, —S(O)₂NR′R″, —NR′SO₂R″, —CN and —NO₂. The number of substituents on a substituted alkyl, substituted heteroalkyl, substituted alkylene, or substituted heteroalkylene group may range from one to (2m′+1), where m′ is the total number of carbon atoms and heteroatoms in such substituted group. The number of substituents on a substituted aryl or substituted heteroaryl group may range from 1 to the total number of open valences on the group.

As used herein, R′, R″, R″′ and R″″ each preferably independently refer to hydrogen, unsubstituted heteroalkyl, unsubstituted aryl, unsubstituted alkyl, alkoxy, thioalkoxy, or unsubstituted arylalkyl groups. When a molecule of the invention includes more than one G group, for example, each of the G groups is independently selected as are each R′, R″, R″′ and R″″ groups when more than one of these groups is present.

As used herein, the term “heteroatom” is meant to include oxygen (O), nitrogen (N), sulfur (S) and silicon (Si). Further, if the term “hetero” immediately precedes a square bracket ([), then any one or more of the groups contained within the square brackets may contain a heteroatom. For example, and without limitation, hetero[(C₁-C₆ alkylene)-(C₆-C₁₂ aryl)] means that the C₁-C₆ alkylene may comprise a heteroatom, the C₆-C₁₂ aryl may comprise a heteroatom or both the C₁-C₆ alkylene and the C₆-C₁₂ aryl may comprise a heteroatom. Similarly, substituted hetero[(C₁-C₆ alkylene)-(C₆-C₁₂ aryl)] means that the C₁-C₆ alkylene may be substituted, the C₆-C₁₂ aryl may be substituted or both the C₁-C₆ alkylene and the C₆-C₁₂ aryl may be substituted.

As used herein, it is to be understood that ranges in the form of Cₓ-C_{y} are inclusive of all of the ranges and sub ranges therein as if individually recited. For example, C₁-C₁₂ includes C₁-C₁₂, C₁-C₁₁, C₁-C₁₀, C₁-C₉, C₁-C₈, C₁-C₇, C₁-C₆, C₁-C₅, C₁-C₄, C₁-C₃, C₁-C₂, C₁-C₁, C₂-C₁₂, C₂-C₁₁, C₂-C₁₀, C₂-C₉, C₂-C₈, C₂-C₇, C₂-C₆, C₂-C₅, C₂-C₄, C₂-C₃, C₂-C₂, C₃-C₁₂, C₃-C₁₁, C₃-C₁₀, C₃-C₉, C₃-C₈, C₃-C₇, C₃-C₆, C₃-C₅, C₃-C₄, C₃-C₃, C₄-C₁₂, C₄-C₁₁, C₄-C₁₀, C₄-C₉, C₄-C₈, C₄-C₇, C₄-C₆, C₄-C₅, C₄-C₄, C₅-C₁₂, C₅-C₁₁, C₅-C₁₀, C₅-C₉, C₅-C₈, C₅-C₇, C₅-C₆, C₅-C₅, C₆-C₁₂, Ce₆—C₁₁, Ce₆—C₁₀, Ce₆—Ce₉, Ce₆—C₈, Ce₆—C₇, Ce₆—Ce₆, C₇-C₁₂, C₇-C₁₁, C₇-C₁₀, C₇-C₉, C₇-C₈, C₇-C₇, C₈-C₁₂, C₈-C₁₁, C₈-C₁₀, C₈-C₉, C₈-C₈, C₉-C₁₂, C₉-C₁₁, C₉-C₁₀, C₉-C₉, C₁₀-C₁₂, C₁₀-C₁₁, C₁₀-C₁₀, C₁₁-C₁₂, C₁₁-C₁₁, and C₁₂-C₁₂.

As used herein, the term “moiety” refers to the radical of a molecule that is attached to another moiety.

As used herein, the symbol

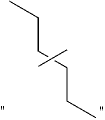

indicates the point at which the displayed moiety is attached to the remainder of the molecule. This is sometimes referred to as a point of attachment. For example, NH₂-(moiety), wherein moiety is

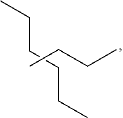

would mean NH₂—CH₂—CH₂—CH₃.

Amino acids are organic compounds that contain amine (—NH₂) and carboxyl (—COOH) functional groups, together with a side chain (sometimes referred to in the art as an R group). As used herein, the term “side chain” when referring to an amino acid (for example “side chain of an amino acid”, or “side chain of a natural amino acid” or “side chain of an unnatural amino acid”) refers to a portion of an amino acid that is not the amine functional portion, not the carboxyl functional portion, and does not include a carbon atom bonded to the amine functional portion or a carbon atom bonded to the carboxyl functional portion of the amino acid. The key elements of an amino acid are carbon (C), hydrogen (H), oxygen (O), and nitrogen (N), although other elements may be found in the side chains of certain amino acids. About 500 naturally occurring amino acids are known and can be classified in many ways. They can be classified according to the core structural functional groups' locations as alpha-, beta-, gamma-, or delta-amino acids. Other categories relate to polarity, pH level, and side chain group type (aliphatic, acyclic, aromatic, containing hydroxyl or sulfur, etc.). Given a particular amino acid a person of skill in the art will readily understand what the “side chain” is and will readily be able to identify if it is a natural amino acid or an unnatural amino acid. See, for example, Dr. Andrew B. Hughes, “Amino Acids, Peptides and Proteins in Organic Chemistry: Origins and Synthesis of Amino Acids, Volume 1”, Wiley-VCH Verlag GmbH & Co. KGaA (2010).

Molecules of the present invention possess asymmetric carbon atoms (optical centers) or double bonds. Unless otherwise clear from context, the racemates, diastereomers, geometric isomers and individual isomers may be encompassed within the scope of the present invention. In compounds and salts of the present invention, there are two carbons denoted by “*”. These two carbons have specific stereochemistry and racemates (and/or other enantiomerically impure mixtures) of these two carbon atoms are specifically excluded from the present invention.

Embodiments of the present invention include a compound having a structure of Formula (I):

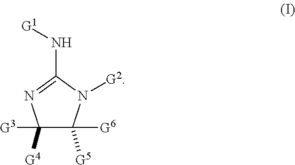

In Formula (I), there are two carbons atoms denoted by “*”. Each of these two carbon atoms are asymmetric carbons and are chiral carbon atoms. In all embodiments of Formula (I) suitable for use in the present invention, both of these two chiral carbon atoms have the same stereoconfiguration when designated using the R and S nomenclature as understood to a person of skill in the art. In other words, if one of the chiral carbon atoms denoted by “*” is in the R configuration, the then other chiral carbon atom denoted by “*” is also in the R configuration. Alternatively if one of the chiral carbon atoms denoted by “*” is in the S configuration, the then other chiral carbon atom denoted by “*” is also in the S configuration. In some illustrative embodiments of the present invention, both of the chiral carbon atoms denoted by “*” are both in the S configuration. In some other illustrative embodiments, both of the chiral carbon atoms denoted by “*” are both in the R configuration. Formula (I) encompasses all of the compounds in Formulae (Ia) and (Ib):

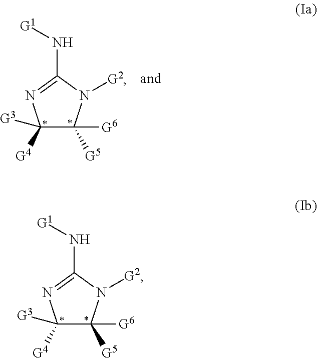

where G¹, G², G³, G⁴, G⁵, and G⁶ are as defined for Formula (I).

In Formulae (I), (Ia) and/or (Ib) G¹ may be a chemical moiety that preferably does not readily react with amino acids. In some embodiments, the G¹ moiety, when considered as a part of Formulae (I), (Ia) and/or (Ib) as a whole, may be a moiety without any charged groups. In other embodiments, the G¹ moiety, when considered as a part of Formulae (I), (Ia) and/or (Ib) as a whole, may be a moiety that has a charge suitable for preferentially selecting a particular amino acid, which amino acid also comprises a charged moiety.

In illustrative embodiments, G¹ may be selected from the group consisting of: H, C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)]. In some illustrative embodiments G¹ may be selected from the group consisting of: H, C₁-C₆ unsubstituted alkyl, C₁-C₆ substituted alkyl, C₆ unsubstituted aryl, C₆ substituted aryl, unsubstituted (C₁-C₆ alkylene)-(C₆ aryl), substituted (C₁-C₆ alkylene)-(C₆ aryl), C₁-C₆ unsubstituted heteroalkyl, C₁-C₆ substituted heteroalkyl, C₆ unsubstituted heteroaryl, C₆ substituted heteroaryl, unsubstituted hetero[(C₁-C₆ alkylene)-(C₆ aryl)], and substituted hetero[(C₁-C₆ alkylene)-(C₆ aryl)]. In some illustrative embodiments, G¹ is not benzyl, substituted benzyl, benzoyl, substituted benzoyl, or —CH₂— cyclohexanyl. In some illustrative embodiments, G¹ is H.

In Formulae (I), (Ia) and/or (Ib) G² may be any chemical moiety. It is preferred that the chemical moiety selected for G² is a moiety that does not readily react with amino acids. In some embodiments, the G² moiety, when considered as a part of Formulae (I), (Ia) and/or (Ib) as a whole, may be a moiety without any charged groups. In other embodiments, the G² moiety, when considered as a part of Formulae (I), (Ia) and/or (Ib) as a whole, may be a moiety that has a charge suitable for preferentially selecting a particular amino acid, which amino acid also comprises a charged moiety.

In some illustrative embodiments, G² may be selected from the group consisting of: H, C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)]. In some illustrative embodiments, G² may be selected from the group consisting of: H, C₁-C₆ unsubstituted alkyl, C₁-C₆ substituted alkyl, C₆ unsubstituted aryl, C₆ substituted aryl, unsubstituted (C₁-C₆ alkylene)-(C₆ aryl), substituted (C₁-C₆ alkylene)-(C₆ aryl), C₁-C₆ unsubstituted heteroalkyl, C₁-C₆ substituted heteroalkyl, C₆ unsubstituted heteroaryl, C₆ substituted heteroaryl, unsubstituted hetero[(C₁-C₆ alkylene)-(C₆ aryl)], and substituted hetero[(C₁-C₆ alkylene)-(C₆ aryl)]. In some illustrative embodiments, G² may be selected from the group consisting of: H, C₁-C₁₂ unsubstituted alkyl, C₁-C₁₂ substituted alkyl, C₆-C₁₂ unsubstituted aryl, C₆-C₁₂ substituted aryl, C₁-C₁₂ unsubstituted heteroalkyl, C₁-C₁₂ substituted heteroalkyl, C₆-C₁₂ unsubstituted heteroaryl, C₆-C₁₂ substituted heteroaryl. In some illustrative embodiments, G² may be selected from the group consisting of: H, C₁-C₆ unsubstituted alkyl, C₁-C₆ substituted alkyl, C₆-C₈ unsubstituted aryl, C₆-C₈ substituted aryl, C₁-C₆ unsubstituted heteroalkyl, C₁-C₆ substituted heteroalkyl, C₅-C₈ unsubstituted heteroaryl, C₅-C₈ substituted heteroaryl. In some illustrative embodiments, G² is H.

In Formulae (I), (Ia) and/or (Ib) G³ may be any chemical moiety. It is preferred that the chemical moiety selected for G³ is a moiety that does not readily react with amino acids. In some embodiments, the G³ moiety, when considered as a part of Formulae (I), (Ia) and/or (Ib) as a whole, may be a moiety without any charged groups. In other embodiments, the G³ moiety, when considered as a part of Formulae (I), (Ia) and/or (Ib) as a whole, may be a moiety that has a charge suitable for preferentially selecting a particular amino acid, which amino acid also comprises a charged moiety.

In some illustrative embodiments, G³ may be selected from the group consisting of: H, C₁-C₁₂ substituted alkyl, C₂-C₁₂ unsubstituted alkyl, C₁-C₁₂ substituted heteroalkyl, and C₁-C₁₂ unsubstituted heteroalkyl. In some illustrative embodiments, G³ may be selected from the group consisting of: H, C₁-C₆ substituted alkyl, C₂-C₆ unsubstituted alkyl, C₁-C₆ substituted heteroalkyl, and C₁-C₆ unsubstituted heteroalkyl. In some illustrative embodiments, G³ is H.

In Formulae (I), (Ia) and/or (Ib), G⁴ may be selected from the group consisting of: unsubstituted C₆-C₁₂ aryl, substituted C₆-C₁₂ aryl, unsubstituted C₆-C₁₂ heteroaryl, substituted C₆-C₁₂ heteroaryl, unsubstituted [(C₆ aryl)-(C₁-C₆ alkyl)-(C₆ aryl)], substituted [(C₆ aryl)-(C₁-C₆ alkyl)-(C₆ aryl)], unsubstituted [(C₆ aryl)-(C₁-C₆ heteroalkyl)-(C₆ aryl)], and substituted [(C₆ aryl)-(C₁-C₆ heteroalkyl)-(C₆ aryl)]. In some illustrative embodiments, G⁴ may be selected from the group consisting of: unsubstituted C₆ aryl, substituted C₆ aryl, unsubstituted C₆ heteroaryl, and substituted C₆ heteroaryl. Often G⁴ is a substituted phenyl group. Often G⁴ is an unsubstituted phenyl group. In some embodiments, G⁴ is a substituted naphthyl group and in other embodiments G⁴ is an unsubstituted naphthyl group. In some embodiments G⁴ is a tri-substituted phenyl group. In some embodiments, G⁴ is a tri-substituted phenyl and is substituted at the para position and substituted at both of the ortho positions.

In Formulae (I), (Ia) and/or (Ib), G⁵ may be selected from the group consisting of: unsubstituted C₆-C₁₂ aryl, substituted C₆-C₁₂ aryl, unsubstituted C₆-C₁₂ heteroaryl, substituted C₆-C₁₂ heteroaryl, unsubstituted [(C₆ aryl)-(C₁-C₆ alkyl)-(C₆ aryl)], substituted [(C₆ aryl)-(C₁-C₆ alkyl)-(C₆ aryl)], unsubstituted [(C₆ aryl)-(C₁-C₆ heteroalkyl)-(C₆ aryl)], and substituted [(C₆ aryl)-(C₁-C₆ heteroalkyl)-(C₆ aryl)]. In some illustrative embodiments, G⁵ may be selected from the group consisting of: unsubstituted C₆ aryl, substituted C₆ aryl, unsubstituted C₆ heteroaryl, and substituted C₆ heteroaryl. Often G⁵ is a substituted phenyl group. Often G⁵ is an unsubstituted phenyl group. In some embodiments, G⁵ is a substituted naphthyl group and in other embodiments G⁵ is an unsubstituted naphthyl group. In some embodiments G⁵ is a tri-substituted phenyl group. In some embodiments, G⁵ is a tri-substituted phenyl and is substituted at the para position and substituted at both of the ortho positions.

In Formulae (I), (Ia) and/or (Ib) G⁶ may be any chemical moiety. It is preferred that the chemical moiety selected for G⁶ is a moiety that does not readily react with amino acids. In some embodiments, the G⁶ moiety, when considered as a part of Formulae (I), (Ia) and/or (Ib) as a whole, may be a moiety without any charged groups. In other embodiments, the G⁶ moiety, when considered as a part of Formulae (I), (Ia) and/or (Ib) as a whole, may be a moiety that has a charge suitable for preferentially selecting a particular amino acid, which amino acid also comprises a charged moiety.

In some illustrative embodiments, G⁶ may be selected from the group consisting of: H, C₁-C₁₂ substituted alkyl, C₂-C₁₂ unsubstituted alkyl, C₁-C₁₂ substituted heteroalkyl, and C₁-C₁₂ unsubstituted heteroalkyl. In some illustrative embodiments, G⁶ may be selected from the group consisting of: H, C₁-C₆ substituted alkyl, C₂-C₆ unsubstituted alkyl, C₁-C₆ substituted heteroalkyl, and C₁-C₆ unsubstituted heteroalkyl. In some illustrative embodiments, G⁶ is H.

Compounds of the present invention having a structure of Formulae (I), (Ia) and/or (Ib) must also conform to all of the following conditions as set out in items (i), (ii), (iii), and (iv):

(i) either:
(a) at least one of G¹, G², G³, and G⁶ is not H; or
(b) at least one of G⁴ and G⁵ is not phenyl; and

(ii) if G⁴ and G⁵ are both

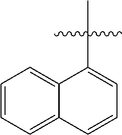 [SMILES: *c1cccc2ccccc12]

or both

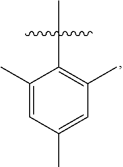

then at least one of G¹, G², G³ and G⁶ is not H; and

(iii) if G³ and G⁶ are both H, and G² is methyl,

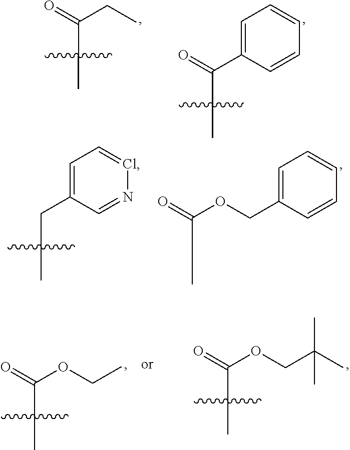

then either G⁴ and G⁵ are not both phenyl, or G¹ is not NO₂, ethyl, tert-butyl, benzyl, cyclohexanyl,

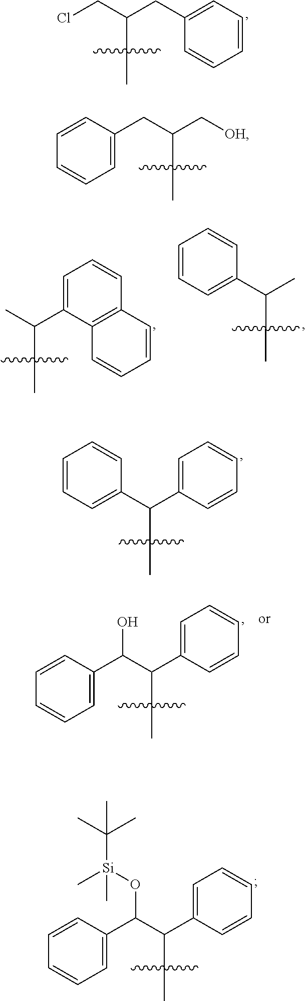

(iv) if G², G³, and G⁶ are all H, and G⁴ and G⁵ are both phenyl, then G¹ is not H, methyl, ethyl, tert-butyl, phenyl, benzyl, cyclohexanyl,

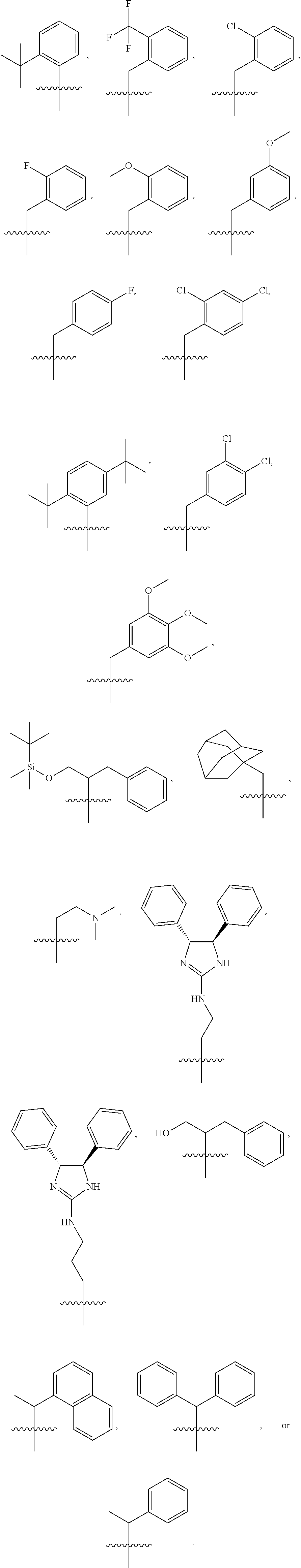

In some illustrative embodiments, compounds of the present invention conform to the conditions (i), (ii), (iii), (iv) as well as the following condition (v):

(v) if G⁴ and G⁵ are both

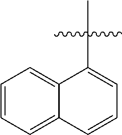 [SMILES: *c1cccc2ccccc12]

or both

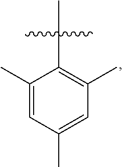

then G¹ is not NO₂, phenyl,

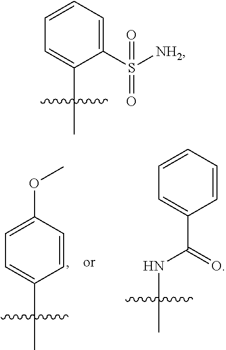

In some illustrative embodiments, compounds of the present invention conform to the conditions (i), (ii), (iii), (iv) as well as the following condition (vi):

(vi) if G³ and G⁶ are both H, and G² is methyl,

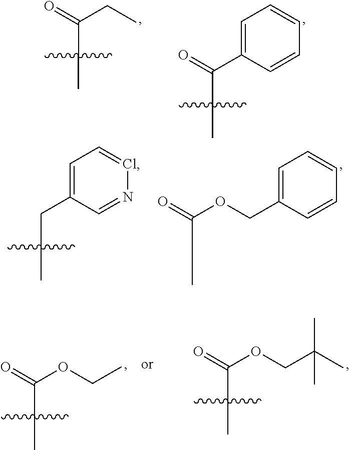

then G¹ is not H.

In some illustrative embodiments, compounds of the present invention conform to the conditions (i), (ii), (iii), (iv), (v), and (vi).

In some illustrative embodiments, compounds of the present invention conform to the conditions (i), (ii), (iii), (iv) as well as the following condition (vii):

(vii) if G², G³, and G⁶ are all H, and G⁴ and G⁵ are both phenyl, then G¹ is not NO₂

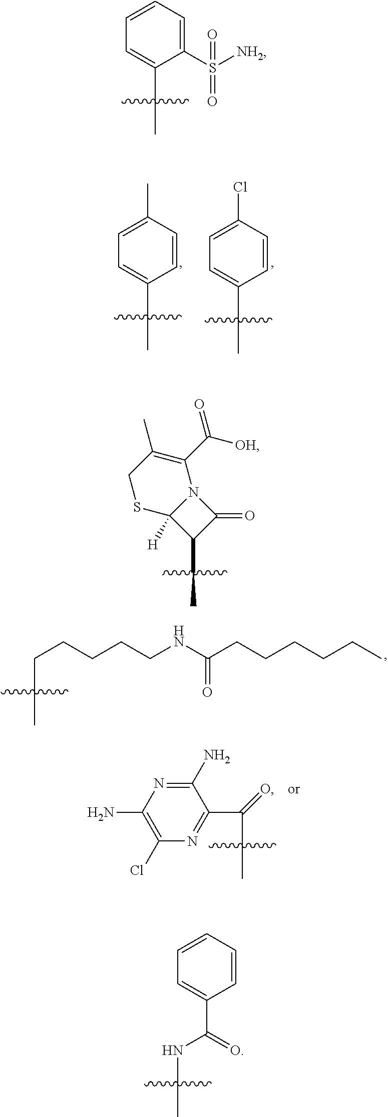

In some illustrative embodiments, compounds of the present invention conform to the conditions (i), (ii), (iii), (iv), (v), and (vii).

In some illustrative embodiments, compounds of the present invention conform to the conditions (i), (ii), (iii), (iv), (vi), and (vii).

In some illustrative embodiments, compounds of the present invention conform to the conditions (i), (ii), (iii), (iv), (v), (vi), and (vii).

In illustrative embodiments, compounds for use in the present invention have no acid moieties and/or groups. In illustrative embodiments, compounds for use in the present invention have no charged moieties and/or groups. In illustrative embodiments, compounds for use in the present invention have neither acid moieties and/or groups nor charged moieties and/or groups.

Compounds of Formulae (I), (Ia) and/or (Ib) may be synthesized using the following general schemes:

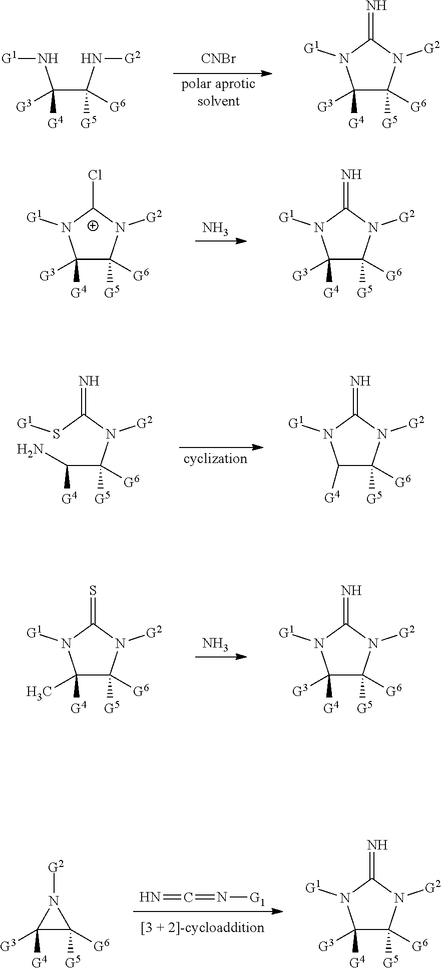

A person of skill in the art will readily be able to adapt the afore-mentioned general schemes to prepare a specific compound of the present invention using this application and the common general knowledge of the art. For example, Wen-Xiong Zhang, Ling Xua and Zhenfeng Xia, “Recent development of synthetic preparation methods for guanidines via transition metal catalysis”, Chem. Commun., 2015, 51, 254-265 provides more details relating to reactions and reaction conditions useful in relation to the general schemes and further some specific examples of how to make some specific compounds of the present invention may be found in the Examples section below.

Compounds of the present invention may be useful in L to D conversion of unactivated alpha-amino acids. In particular, a solubility-induced diastereomeric transformation (SIDT) strategy involving compounds of Formulae (I), (Ia) and/or (Ib) converts amino acids from one form, a racemate, or other mixture of D and L forms to another, single form D enantiomer or single form L enantiomer. An exemplification, without limitation, of this mechanism is illustrated generally in Scheme 1 below.

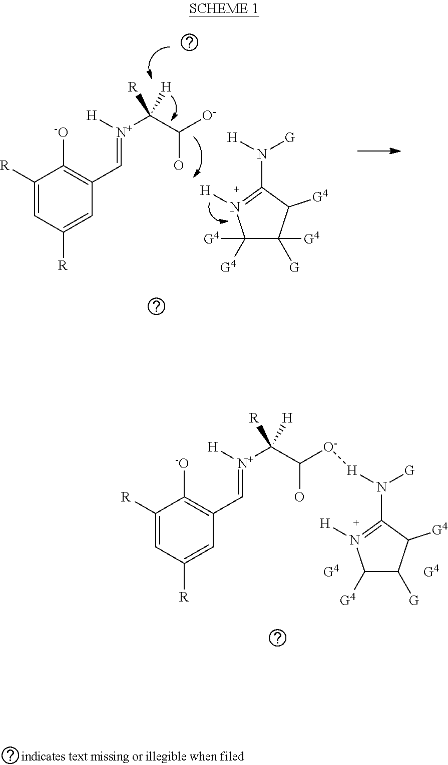

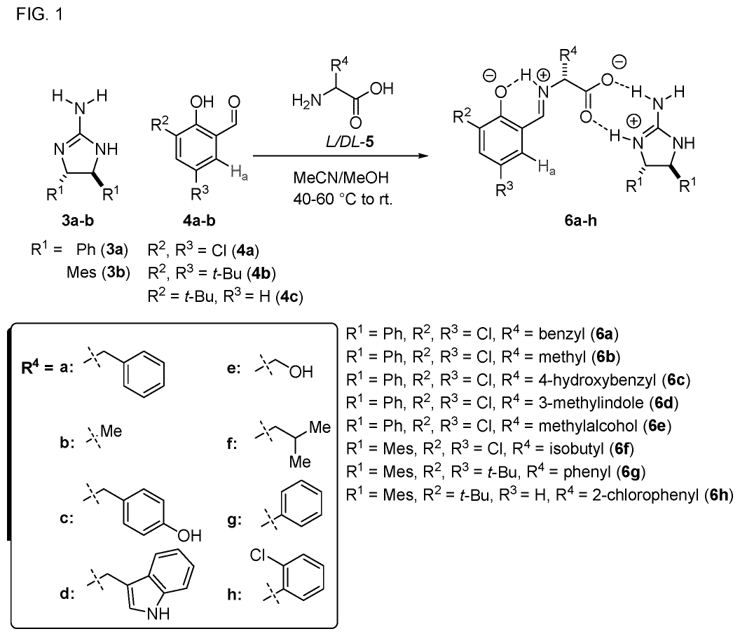

Ternary complexes of an alpha-amino acid with a salicylaldehyde derivative and a chiral guanidine, such as those set out in Formulae (I), (Ia) and/or (Ib), may be obtained in good yield as diastereomerically pure imino acid salt complexes which may then be hydrolysed to obtain enantiopure alpha-amino acids. SIDT relies on small solubility difference between equilibrating diastereomers. Deracemisation of free unactivated amino acids may be challenging due to the high pKₐ of the alpha-proton. Using two types of strong hydrogen bonds in concert, the present invention enables deracemisation of unactivated amino acids using SIDT under mild reaction conditions (See FIG. 1). The same method may be used to convert readily available L amino acids to D amino acids and vice-versa as well as the deracemisation of racemic (or other enantiomerically impure mixtures) natural and unnatural amino acids.

Organic SIDT can be used for deracemisation of free, unprotected amino acids without the need for developing stereoselective receptors. A single system consisting of a salicylaldehyde derivative and a chiral guanidine may be used to detect the D/L ratio as well as to carry out L to D conversion (or alternatively the D to L conversion) of a variety of amino acids in a unified way. X-ray and computational data indicate that two special types of strong hydrogen bonds are involved in facilitating rapid racemisation of amino acids which is beneficial for SIDT. Excellent diastereoselectivity (up to >100:2 dr) is observed with the same sense of stereoselectivity for the substrates and subsequent hydrolysis of the imino acid salts results in enantiopure amino acids (up to >98% ee). The present invention provides an operationally simple method as an attractive strategy towards the synthesis of enantiomerically pure amino acids, including D-amino acids.

The difference in solubility between the D-forms and the L-forms may be exploited in combination with a racemization to achieve a dynamic kinetic resolution of unprotected alpha-amino acids. When one equivalent of amino acid is stirred in the presence of a salicylaldehyde derivative and an excess of a compound of Formulae (I), (la) and/or (Ib), the first equivalent of the compound of Formulae (I), (la) and/or (Ib) may be used for chiral salt formation while the excess acts as a general base catalyst to epimerize the imino acid guanidinium salt. A small excess of salicylaldehyde derivative may be used over the amino acid to ensure that there is no free amino acid remaining in solution. Free amino acids cannot be readily epimerized and thus would not undergo L to D conversion or D to L conversion, resulting in a decrease in enantiopurity of the amino acid product.

The ratio of (S,S)-D and (S,S)-L in solution may be determined by ¹H NMR spectroscopy by integrating the Hₐ signals of the two diastereomeric salts as set out in Scheme 2 below:

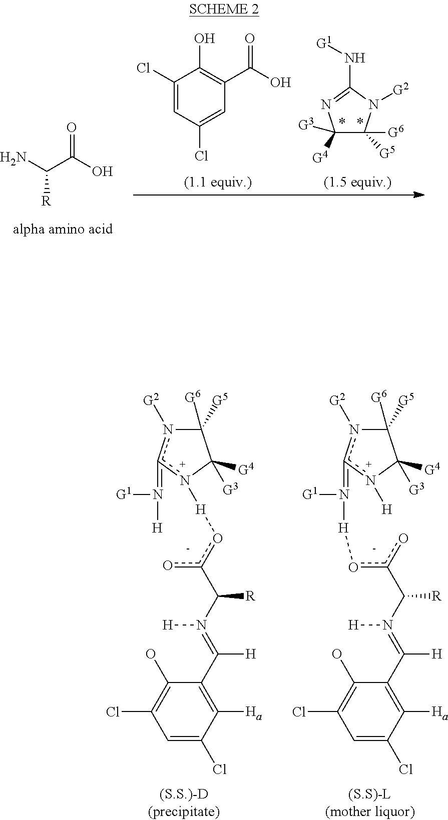

There are approximately equal concentrations of (S,S)-D and (S,S)-L in the initial reaction mixture. The mixture is heated in methanol for a few hours to ensure quantitative formation of the imino acid salt. Afterwards, the methanol is evaporated and a solvent is added wherein the two diastereomeric salts have significantly different solubility properties (for example, MeCN or THF may be the added solvent), and overtime, the less soluble (S,S)-D imino acid quanidinium salt begins to precipitate out of the solution. As the (S,S)-D salt precipitates out of solution, the diastereomeric ratio in solution re-equilibrates forming more of the (S,S)-D salt which induces further precipitation, resulting in high yields.

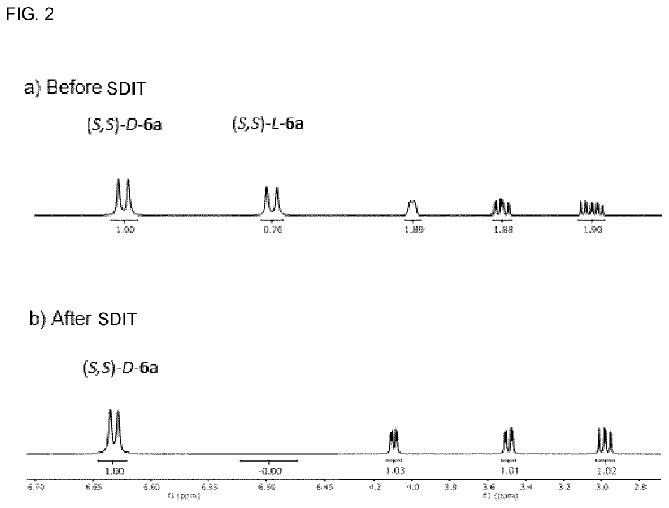

The ¹H NMR spectrum of the precipitated salt showed that the Hₐ signal due to (S,S)-L salt has essentially disappeared (<1%) and the precipitate is entirely the (S,S)-D salt (FIG. 2). The diastereomerically pure imino acid guanidinium salt may then be hydrolysed by aqueous acid to obtain the enantiomerically pure amino acid while the chiral guanidinium that remains in solution may be recycled for further use.

The Hₐ signal for (S,S)-D appears more downfield than (S,S)-L and this provides an opportunity to determine the absolute configuration and enantiomeric excess of amino acids. In addition to the Hₐ signal, other ¹H NMR signals such as the alpha-proton or the beta-proton may be used to determine the ratio of (S,S)-D and (S,S)-L. There appears to be an agreement between the integration ratios obtained from Hₐ signals with other ¹H NMR signals.

Some alpha amino acids present some challenges. The thiol group of cysteine may intramolecularly attach the imine carbon leading to an undesired product. Beta-branched amino acids, such as valine, have additional steric bulk around the alpha-carbon which may impede epimerization of the alpha-position under mild conditions. Further, if the alpha-position of L-threonine and L-isoleucine were to be readily epimerizable under mild conditions, then only the D-allo isomers may be formed since full racemisation of both stereocentres may not be possible. Arginine may also present some challenges since the guanidine group of arginine may compete with the chiral guanidine for guanidinium salt formation, which may reduce purity. Proline is also challenging since secondary amines may not form the imine functionality thereby reducing facile epimerization. Nevertheless and notwithstanding these challenges, enhanced stereoselective purity may still be obtained for some of these challenging amino acids.

An important factor in successful SIDT is that the two diastereomeric salts have sufficient differences in its solubility properties. The compounds of the present invention may provide a solubility difference of up to or over 30 times (maximum concentration of 0.48 M vs 0.015 M) of one diastereomer over the other. This may improve precipitation and deracemisation. Further, particular compounds of the present invention may be better suited to deracemizing one particular amino acid or one particular type of amino acid (e.g. positively charged, or negatively charged, etc.) when compared to other chiral guanidines. Many chiral guanidines previously disclosed provide negligible solubility differences, resulting in their diastereomeric salts not precipitating out of solution but rather resulting in both diastereomers “oiling out” of solution as a viscous brown oil, effectively resulting in limited to zero deracemization. The particular suitability properties of the diastereomeric salts of a particular chiral guanidine with respect to a particular amino acid is variable and may lead to efficiencies being found with particular pairings of chiral guanidines and amino acids.

Salts of the present invention may comprise a structure of Formula II:

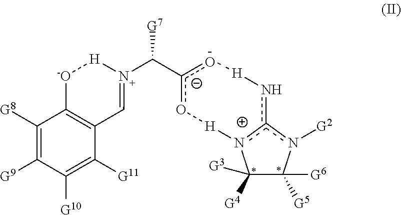

In Formula (II), there are two carbons atoms denoted by “*”. Each of these two carbon atoms are asymmetric carbons and are chiral carbon atoms. In all embodiments of Formula (II) suitable for use in the present invention, both of these two chiral carbon atoms have the same stereoconfiguration when designated using the R and S nomenclature as understood to a person of skill in the art. In other words, if one of the chiral carbon atoms denoted by “*” is in the R configuration, the then other chiral carbon atom denoted by “*” is also in the R configuration. Alternatively if one of the chiral carbon atoms denoted by “*” is in the S configuration, the then other chiral carbon atom denoted by “*” is also in the S configuration. In some illustrative embodiments of the present invention, both of the chiral carbon atoms denoted by “*” are both in the S configuration. In some other illustrative embodiments, both of the chiral carbon atoms denoted by “*” are both in the R configuration. Formula (II) encompasses all of the compounds in Formulae (IIa) and (IIb):

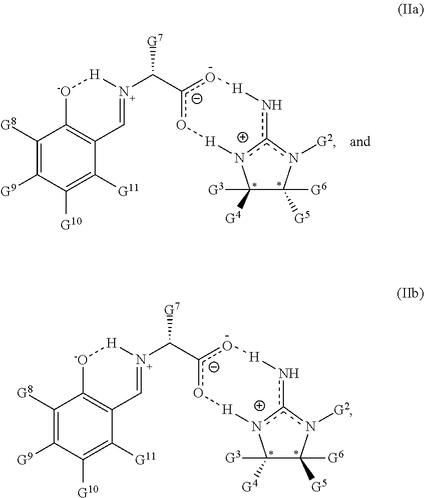

where G², G³, G⁴, G⁵, G⁶, G⁷, G⁸, G⁹, G¹⁰, and G¹¹ are as defined for Formula (II).

Further, in salts of any one of Formulae (II), (IIa), and/or (IIb) there are some charged moieties in the following portion of the molecule:

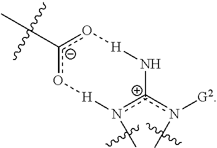

The charge of and/or in this moiety is dynamic. In this charged moiety, the negative charge is delocalized between the two oxygen atoms and the positive charge is delocalized between the three nitrogen atoms. The charge of this moiety may also be illustrated using an alternative such as:

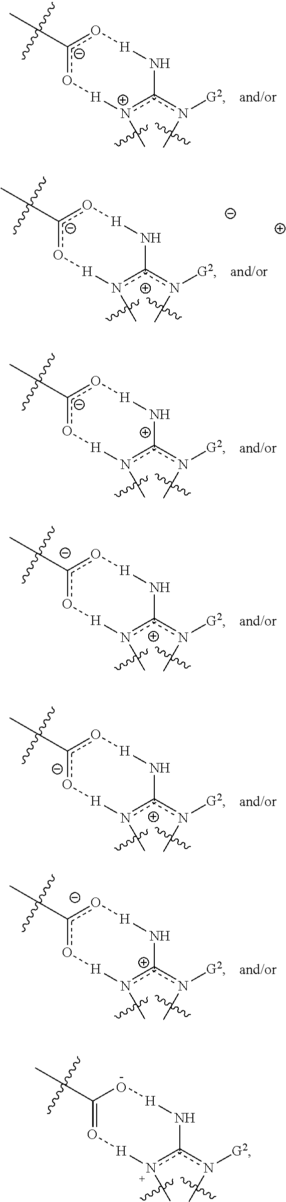

etc.
The use of the drawings and/or their alternatives representing this charged portion of salts of the present invention are interchangeable and mean the same as used herein. A person skilled in the art of chemistry will readily understand the dynamic state of the charge in this moiety.

In Formulae (II), (IIa) and/or (IIb) G² may be any chemical moiety. It is preferred that the chemical moiety selected for G² is a moiety that does not readily react with amino acids. In some embodiments, the G² moiety, when considered as a part of Formulae (II), (IIa) and/or (IIb) as a whole, may be a moiety without any charged groups. In other embodiments, the G² moiety, when considered as a part of Formulae (II), (IIa) and/or (IIb) as a whole, may be a moiety that has a charge suitable for preferentially selecting a particular amino acid, which amino acid also comprises a charged moiety.

In some illustrative embodiments, G² may be selected from the group consisting of: H, C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)]. In some illustrative embodiments, G² may be selected from the group consisting of: H, C₁-C₆ unsubstituted alkyl, C₁-C₆ substituted alkyl, C₆ unsubstituted aryl, C₆ substituted aryl, unsubstituted (C₁-C₆ alkylene)-(C₆ aryl), substituted (C₁-C₆ alkylene)-(C₆ aryl), C₁-C₆ unsubstituted heteroalkyl, C₁-C₆ substituted heteroalkyl, C₆ unsubstituted heteroaryl, C₆ substituted heteroaryl, unsubstituted hetero[(C₁-C₆ alkylene)-(C₆ aryl)], and substituted hetero[(C₁-C₆ alkylene)-(C₆ aryl)]. In some illustrative embodiments, G² may be selected from the group consisting of: H, C₁-C₁₂ unsubstituted alkyl, C₁-C₁₂ substituted alkyl, C₆-C₁₂ unsubstituted aryl, C₆-C₁₂ substituted aryl, C₁-C₁₂ unsubstituted heteroalkyl, C₁-C₁₂ substituted heteroalkyl, C₆-C₁₂ unsubstituted heteroaryl, C₆-C₁₂ substituted heteroaryl. In some illustrative embodiments, G² may be selected from the group consisting of: H, C₁-C₆ unsubstituted alkyl, C₁-C₆ substituted alkyl, C₆-C₈ unsubstituted aryl, C₆-C₈ substituted aryl, C₁-C₆ unsubstituted heteroalkyl, C₁-C₆ substituted heteroalkyl, C₅-C₈ unsubstituted heteroaryl, C₅-C₈ substituted heteroaryl. In some illustrative embodiments, G² is H.

In Formulae (II), (IIa) and/or (IIb) G³ may be any chemical moiety. It is preferred that the chemical moiety selected for G³ is a moiety that does not readily react with amino acids. In some embodiments, the G³ moiety, when considered as a part of Formulae (II), (IIa) and/or (IIb) as a whole, may be a moiety without any charged groups. In other embodiments, the G³ moiety, when considered as a part of (II), (IIa) and/or (IIb) as a whole, may be a moiety that has a charge suitable for preferentially selecting a particular amino acid, which amino acid also comprises a charged moiety.

In some illustrative embodiments, G³ may be selected from the group consisting of: H, C₁-C₁₂ substituted alkyl, C₂-C₁₂ unsubstituted alkyl, C₁-C₁₂ substituted heteroalkyl, and C₁-C₁₂ unsubstituted heteroalkyl. In some illustrative embodiments, G³ may be selected from the group consisting of: H, C₁-C₆ substituted alkyl, C₂-C₆ unsubstituted alkyl, C₁-C₆ substituted heteroalkyl, and C₁-C₆ unsubstituted heteroalkyl. In some illustrative embodiments, G³ is H.

In Formulae (II), (IIa) and/or (IIb), G⁴ may be selected from the group consisting of: unsubstituted C₆-C₁₂ aryl, substituted C₆-C₁₂ aryl, unsubstituted C₆-C₁₂ heteroaryl, substituted C₆-C₁₂ heteroaryl, unsubstituted [(C₆ aryl)-(C₁-C₆ alkyl)-(C₆ aryl)], substituted [(C₆ aryl)-(C₁-C₆ alkyl)-(C₆ aryl)], unsubstituted [(C₆ aryl)-(C₁-C₆ heteroalkyl)-(C₆ aryl)], and substituted [(C₆ aryl)-(C₁-C₆ heteroalkyl)-(C₆ aryl)]. In some illustrative embodiments, G⁴ may be selected from the group consisting of: unsubstituted C₆ aryl, substituted C₆ aryl, unsubstituted C₆ heteroaryl, and substituted C₆ heteroaryl. Often G⁴ is a substituted phenyl group. Often G⁴ is an unsubstituted phenyl group. In some embodiments, G⁴ is a substituted naphthyl group and in other embodiments G⁴ is an unsubstituted naphthyl group. In some embodiments G⁴ is a tri-substituted phenyl group. In some embodiments, G⁴ is a tri-substituted phenyl and is substituted at the para position and substituted at both of the ortho positions.

In Formulae (II), (IIa) and/or (IIb), G⁵ may be selected from the group consisting of: unsubstituted C₆-C₁₂ aryl, substituted C₆-C₁₂ aryl, unsubstituted C₆-C₁₂ heteroaryl, substituted C₆-C₁₂ heteroaryl, unsubstituted [(C₆ aryl)-(C₁-C₆ alkyl)-(C₆ aryl)], substituted [(C₆ aryl)-(C₁-C₆ alkyl)-(C₆ aryl)], unsubstituted [(C₆ aryl)-(C₁-C₆ heteroalkyl)-(C₆ aryl)], and substituted [(C₆ aryl)-(C₁-C₆ heteroalkyl)-(C₆ aryl)]. In some illustrative embodiments, G⁵ may be selected from the group consisting of: unsubstituted C₆ aryl, substituted C₆ aryl, unsubstituted C₆ heteroaryl, and substituted C₆ heteroaryl. Often G⁵ is a substituted phenyl group. Often G⁵ is an unsubstituted phenyl group. In some embodiments, G⁵ is a substituted naphthyl group and in other embodiments G⁵ is an unsubstituted naphthyl group. In some embodiments G⁵ is a tri-substituted phenyl group. In some embodiments, G⁵ is a tri-substituted phenyl and is substituted at the para position and substituted at both of the ortho positions.

In Formulae (II), (IIa) and/or (IIb) G⁶ may be any chemical moiety. It is preferred that the chemical moiety selected for G⁶ is a moiety that does not readily react with amino acids. In some embodiments, the G⁶ moiety, when considered as a part of Formulae (I), (la) and/or (Ib) as a whole, may be a moiety without any charged groups. In other embodiments, the G⁶ moiety, when considered as a part of Formulae (I), (la) and/or (Ib) as a whole, may be a moiety that has a charge suitable for preferentially selecting a particular amino acid, which amino acid also comprises a charged moiety.

In some illustrative embodiments, G⁶ may be selected from the group consisting of: H, C₁-C₁₂ substituted alkyl, C₂-C₁₂ unsubstituted alkyl, C₁-C₁₂ substituted heteroalkyl, and C₁-C₁₂ unsubstituted heteroalkyl. In some illustrative embodiments, G⁶ may be selected from the group consisting of: H, C₁-C₆ substituted alkyl, C₂-C₆ unsubstituted alkyl, C₁-C₆ substituted heteroalkyl, and C₁-C₆ unsubstituted heteroalkyl. In some illustrative embodiments, G⁶ is H.

In Formulae (II), (IIa) and/or (IIb) G⁷ may be any chemical moiety. In some illustrative embodiments, G⁷ is selected from the group consisting of: C₁-C₁₂ unsubstituted alkyl, C₁-C₁₂ substituted alkyl, C₆-C₁₂ unsubstituted aryl, C₆—C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)]. In some illustrative embodiments, G⁷ is a side chain of a natural amino acid or a side chain of an unnatural amino acid. In some illustrative embodiments, G⁷ is selected from the group consisting of: CH_{3, 4}-hydroxyphenyl, CH₂OH, propan-2-yl, phenyl, 2-chlorophenyl, CH₂-phenyl, CH₂—CH₂—S—CH₃, butan-2-yl, CH₂-3-H-indole, CH₂-1,3-benxodioxole, CH₂-2,3,4,5,6-pentachloro-phenyl, CH₂-4-nitro-phenyl, CH₂-4-fluoro-phenyl, CH₂-4-bromo-phenyl, CH₂-4-iodo-phenyl, CH₂-cyclohexane, CH₂-naphthyl, CH₂-cylopentane, CH₂-ethynyl, CH₂—C(═CH₂)(CH₃), CH₂-3-H-pyrrole, CH₂—CH₂-phenyl, CH₂-3-H,7-hydroxy-indole, CH₂-3-H,6-hydroxy-indole, and CH₂-4-azido-phenyl.

In Formulae (II), (IIa) and/or (IIb) each of G⁸, G⁹, G¹⁰, and G¹¹ are independently selected from any chemical moiety. In illustrative embodiments, each of G⁸, G⁹, G¹⁰, and G¹¹ may independently be selected from the group consisting of: H, halogen, C₁-C₁₂ alkyl, C₆-C₁₂ aryl, C₁-C₁₂ O-alkyl, C₆-C₁₂ O-aryl, C₁-C₁₂ N-dialkyl and C₆-C₁₂ N-diaryl. In some illustrative embodiments, each of G⁸, G⁹, G¹⁰, and G¹¹ may independently be selected from the group consisting of: H, halogen, C₁-C₁₂ alkyl, C₆-C₁₂ aryl. In some illustrative embodiments, each of G⁸, G⁹, G¹⁰, and G¹¹ may independently be selected from the group consisting of: H, halogen, C₁-C₁₂ alkyl. In some illustrative embodiments, G⁸, G⁹, G¹⁰, and G¹¹ may independently be H, Cl, and tert-butyl (t-Bu). In some illustrative embodiments, G⁸ is selected from the group consisting of: H, chloro, and tert-butyl. In some illustrative embodiments, G⁸ is selected from the group consisting of: chloro, and tert-butyl. In some illustrative embodiments, G⁹ is H. In some illustrative embodiments, G¹⁰ is selected from the group consisting of: H, chloro, and tert-butyl. In some illustrative embodiments, G¹¹ is H.

Explicitly excluded from compounds and salts of the present invention and from salts of Formulae (II), (IIa) and/or (IIb) are the following:

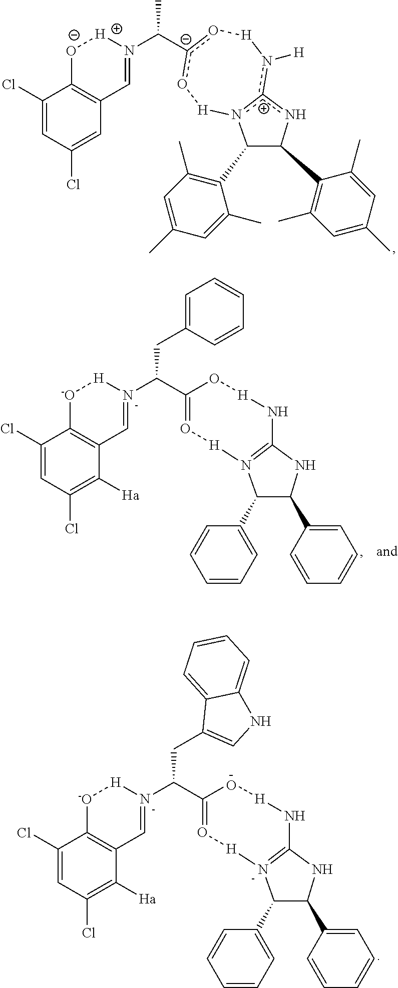

Imino Acid Guandinium Salts of the present invention may be prepared using the following general procedure. To solution of salicylaldehyde derivative (1.1 equiv.) and chiral guanidine derivative (1.5 equiv.) dissolved in acetonitrile or methanol (0.2 M) was added amino acid (1.0 equiv.) at 40° C. and stirred for 4 hours. More specific procedures for making specific compounds may be found elsewhere in this description, for example, in the Examples section. The following compounds are exemplary of compounds suitable for preparation using this general procedure:

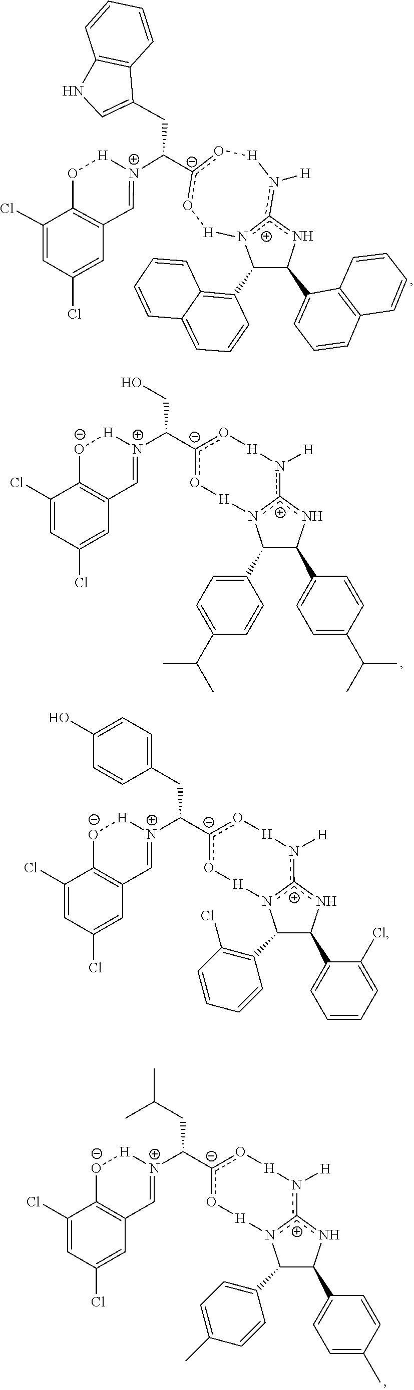

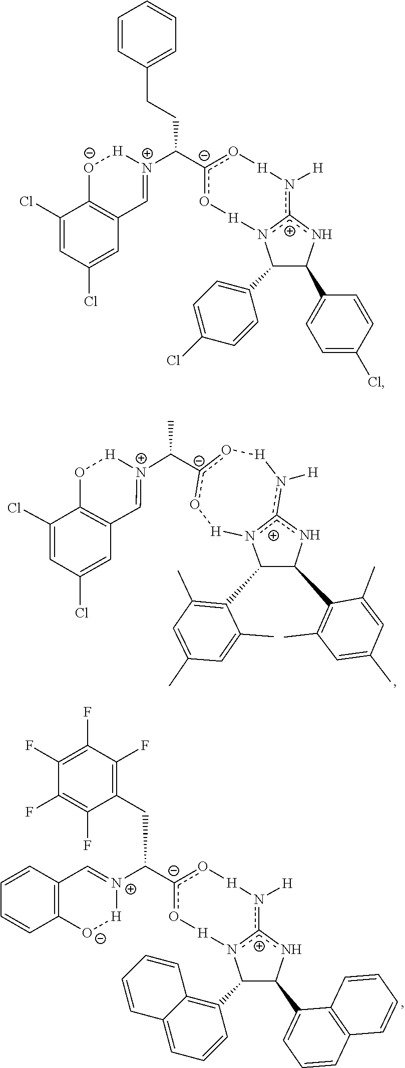

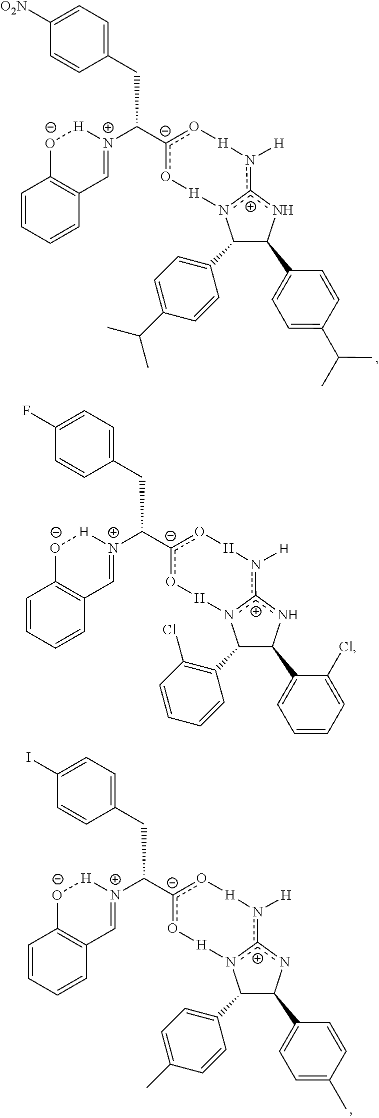

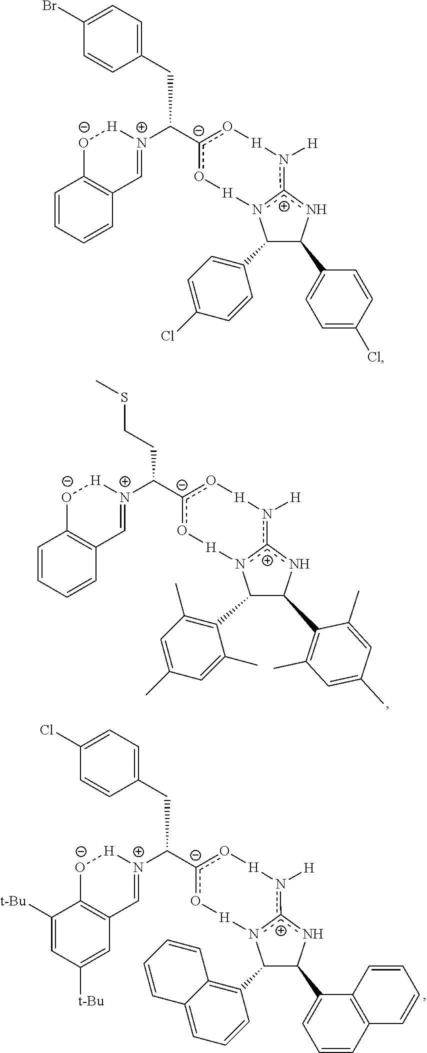

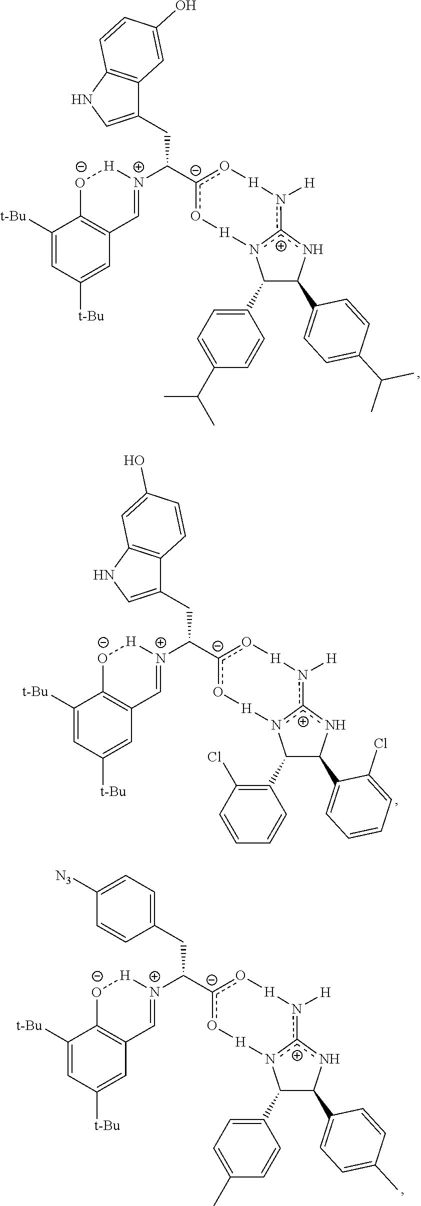

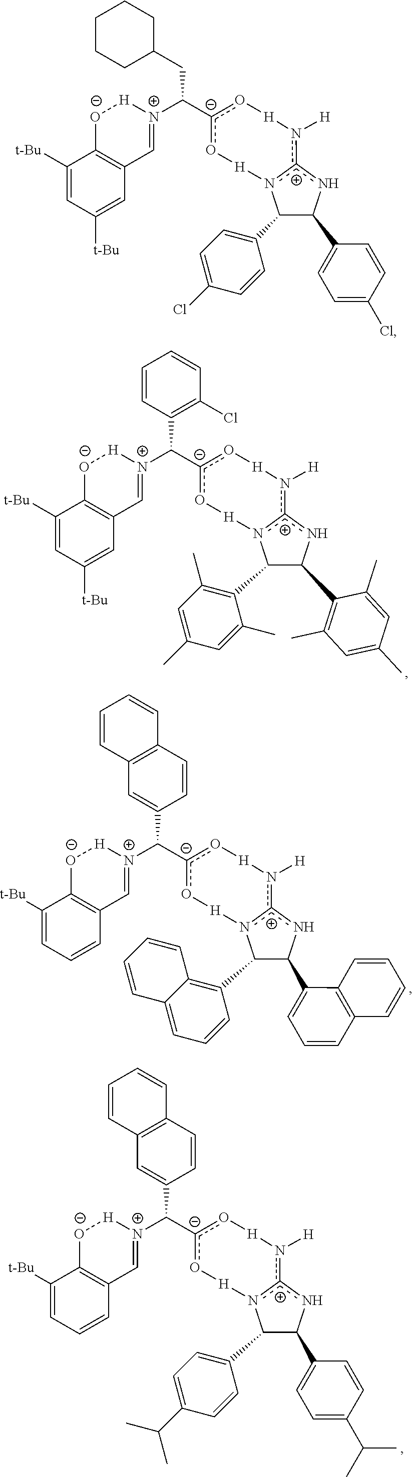

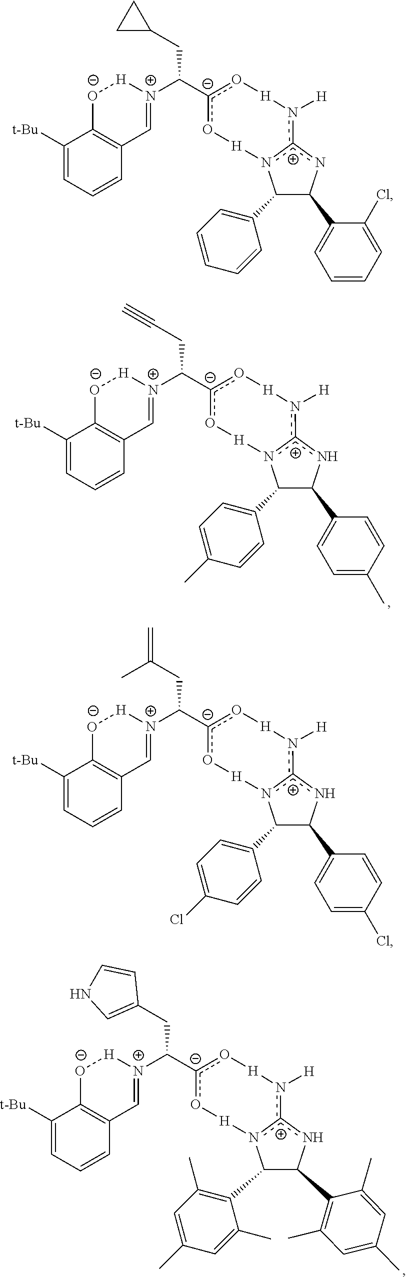

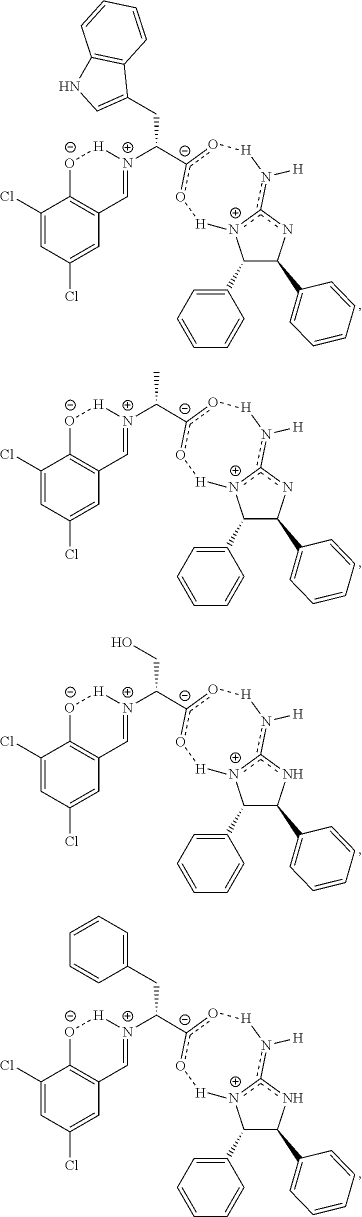

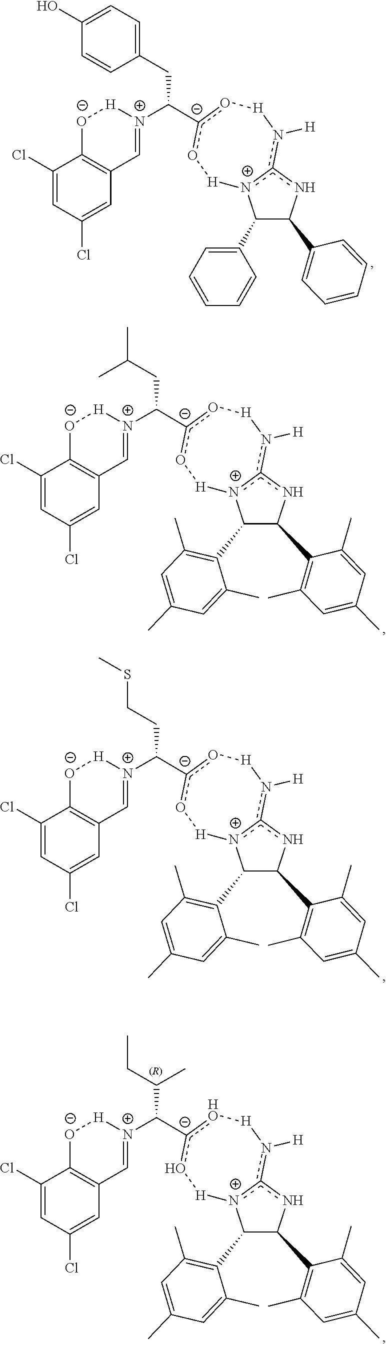

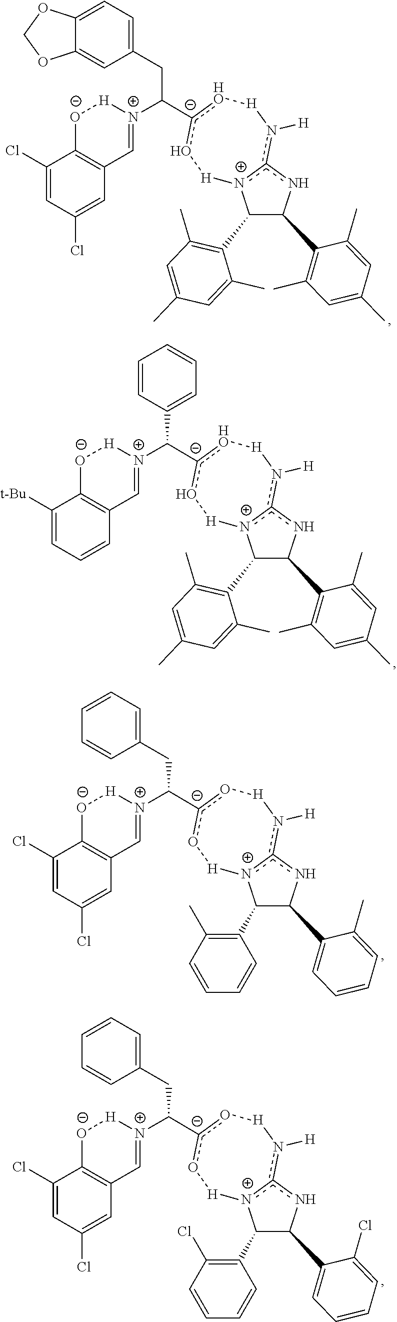

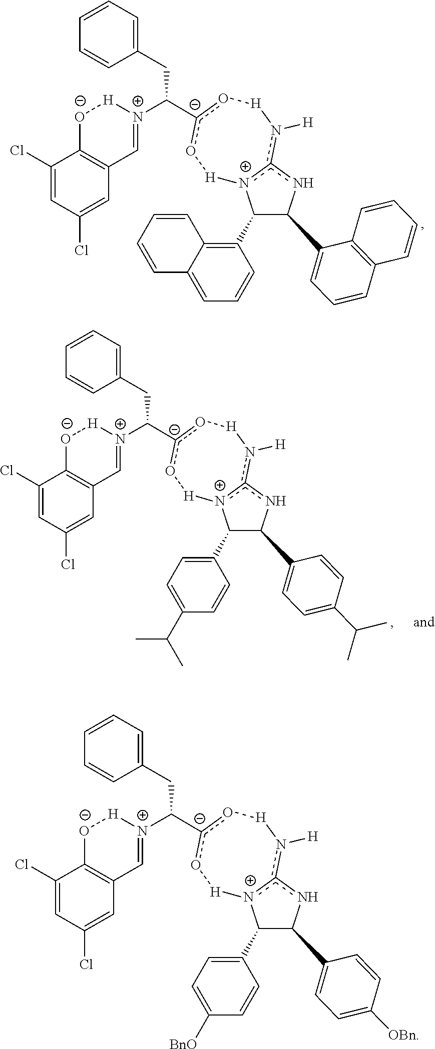

### EXAMPLES

The following examples are illustrative of some of the embodiments of the invention described herein. These examples do not limit the spirit or scope of the invention in any way.

### Example 1: 4-Chloro DPEN Guanidine

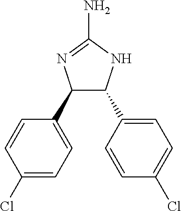 [SMILES: NC1=N[C@H](c2ccc(Cl)cc2)[C@@H](c2ccc(Cl)cc2)N1]

To a stirred solution of 4-CI-DPEN diamine (1.5 g, 5.33 mmol) in CHCl₃ (48 mL) was slowly added CNBr (0.68 g, 6.4 mmol, 1.2 equiv.) in 5 mL CHCl₃ at 0° C. After 0.5 h at 0° C., the mixture was allowed to room temperature, stirred for overnight, and concentrated under reduced pressure. The guanidinium salt was then basified by dissolving in THF (30 mL)/H₂O (40 mL) and adding NaOH (320 mg, 8 mmol). The reaction mixture was stirred for 1 h, extracted with CHCl₃, dried over MgSO₄, and concentrated to provide 4-Chloro DPEN Guanidine.

### Example 2: 2-Chloro DPEN Guanidine

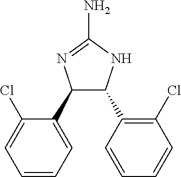 [SMILES: NC1=N[C@H](c2ccccc2Cl)[C@@H](c2ccccc2Cl)N1]

To a stirred solution of 2-CI-DPEN diamine (5.56 g, 20 mmol) in CHCl₃ (180 mL) was slowly added CNBr (2.3 g, 22 mmol) in 10 mL CHCl₃ at 0° C. After 0.5 h at 0° C., the mixture was allowed to room temperature, stirred for overnight, and concentrated under reduced pressure. The guanidinium salt was then basified by dissolving in CHCl₃ (50 mL)/H₂O (50 mL) and adding KOH (1.66 g, 29 mmol). The reaction mixture was stirred for 1 h and CHCl₃ was concentrated under reduced pressure. The reaction mixture was filtered, washed with H₂O, and dried with house vacuum to yield 2-Chloro DPEN Guanidine.

### Example 3: DPEN Guanidine

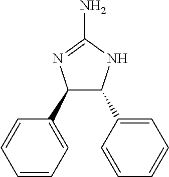 [SMILES: NC1=N[C@H](c2ccccc2)[C@@H](c2ccccc2)N1]

To a stirred solution of DPEN diamine (3 g, 14 mmol) in CHCl₃ (130 mL) was slowly added CNBr (1.8 g, 17 mmol) in 10 mL CHCl₃ at 0° C. After 0.5 h at 0° C., the mixture was allowed to room temperature, stirred for overnight, and concentrated under reduced pressure. The guanidinium salt was then basified by dissolving in CHCl₃ (40 mL)/H₂O (40 mL) and adding NaOH (0.8 g). The reaction mixture was stirred for 1 h, extracted with CHCl₃, dried over MgSO₄, and concentrated to yield DPEN Guanidine.

### Example 4: Mesityl Guanidine

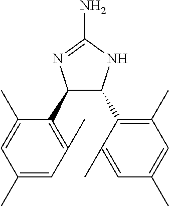 [SMILES: Cc1cc(C)c([C@H]2N=C(N)N[C@@H]2c2c(C)cc(C)cc2C)c(C)c1]

To a stirred suspension of mesityl diamine-2HCl (2.8 g, 7.6 mmol) in H₂O (50 mL) was added NaOH (0.76 g, 19 mmol) at room temperature, stirred for 1 h, and filtered. To a stirred solution of mesityl diamine (2.25 g, 7.6 mmol) in CHCl₃ (70 mL) was slowly added CNBr (1.04 g, 9.9 mmol) in 10 mL CHCl₃ at 0° C. After 0.5 h at 0° C., the mixture was allowed to room temperature, stirred for overnight, and concentrated under reduced pressure. The guanidinium salt was then basified by dissolving in CHCl₃ (30 mL)/H₂O (30 mL) and adding NaOH (320 mg). The reaction mixture was stirred for 1 h and CHCl₃ was concentrated under reduced pressure. The reaction mixture was filtered, washed with H₂O, and dried with house vacuum to yield Mesityl Guanidine.

### Example 5: 4-OBn DPEN Guanidine

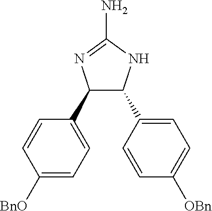 [SMILES: NC1=N[C@H](c2ccc(OCc3ccccc3)cc2)[C@@H](c2ccc(OCc3ccccc3)cc2)N1]

To a stirred solution of 4-OBn-DPEN diamine (3.7 g, 8.7 mmol) in CHCl₃(70 mL)/THF (80 mL) was slowly added CNBr (1.2 g, 11.3 mmol) in 10 mL CHCl₃ at 0° C. After 0.5 h at 0° C., the mixture was allowed to room temperature, stirred for overnight, and concentrated under reduced pressure. The guanidinium salt (1.1 mmol) was then basified by dissolving in 95% EtOH(5.5 mL) and adding KOH (130 mg, 2.3 mmol). The reaction mixture was stirred for 1 h at 50° C. and concentrated to provide the crude guanidine. The residue was added H₂O, filtered, and dried with house vacuum to yield 4-OBn DPEN Guanidine.

### Example 6: 4-Isopropyl DPEN Guanidine

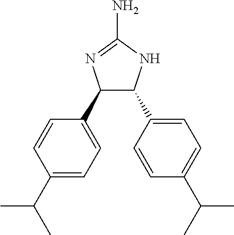 [SMILES: CC(C)c1ccc([C@H]2N=C(N)N[C@@H]2c2ccc(C(C)C)cc2)cc1]

To a stirred suspension of 4-Isopropyl diamine-2HCl (5 g, 13.5 mmol) in H₂O (50 mL) was added KOH (2.27 g, 41 mmol) at room temperature, stirred for 1 h. The reaction mixture was extracted with CHCl₃, dried over MgSO₄, and concentrated under reduced pressure. To a stirred solution of 4-Isopropyl diamine (4 g, 13.5 mmol) in CHCl₃ (120 mL) was slowly added CNBr (1.86 g, 17.6 mmol) in 10 mL CHCl₃ at 0° C. After 0.5 h at 0° C., the mixture was allowed to room temperature, stirred for overnight, and concentrated under reduced pressure to yield 4-Isopropyl DPEN Guanidine.

### Example 7: 4-Methyl DPEN Guanidine

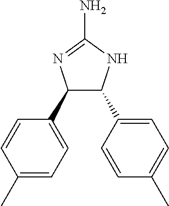 [SMILES: Cc1ccc([C@H]2N=C(N)N[C@@H]2c2ccc(C)cc2)cc1]

To a stirred solution of 4-Me-DPEN diamine (3.4 g, 14 mmol) in CHCl₃ (120 mL) was slowly added CNBr (1.9 g, 18 mmol) in 20 mL CHCl₃ at 0° C. After 0.5 h at 0° C., the mixture was allowed to room temperature, stirred for overnight, and concentrated under reduced pressure. The guanidinium salt was then basified by dissolving in CHCl₃ (50 mL)/H₂O (50 mL) and adding KOH (1.4 g, 25 mmol). The reaction mixture was stirred for 1 h and CHCl₃ was concentrated under reduced pressure. The reaction mixture was filtered, washed with H₂O, and dried with house vacuum to yield 4-Methyl DPEN Guanidine.

### Example 8: Naphthyl Guanidine

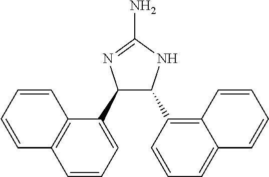 [SMILES: NC1=N[C@H](c2cccc3ccccc23)[C@@H](c2cccc3ccccc23)N1]

To a stirred solution of bis-1-naphthyl diamine-2HCl (15 g, 39 mmol) in H₂O (400 mL) was added NaOH (4.7 g, 117 mmol) at room temperature, stirred for 1 h, and extracted with CHCl₃. The mixture was dried over MgSO₄ and concentrated under reduced pressure. To a stirred solution of diamine (10 g, 32 mmol) in CHCl₃ (380 mL) was slowly added CNBr (4.07 g, 38.4 mmol) in 20 mL CHCl₃ at 0° C. After 0.5 h at 0° C., the mixture was allowed to room temperature, stirred for 3 h, and concentrated under reduced pressure. The guanidinium salt (14 g, 33.4 mmol) was then basified by dissolving in MeOH (250 mL)/H₂O (100 mL) and adding NaOH (2.67 g, 66.8 mmol). The reaction mixture was stirred for 3 h and methanol was concentrated under reduced pressure. The reaction mixture was filtered, washed with H₂O, and dried with house vacuum to yield Naphthyl Guanidine.

### Example 9

| Code Name as | |
|---|---|
| used herein | Structure |
| (S,S)-3a | 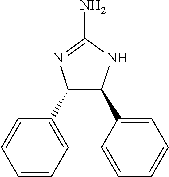 [SMILES: NC1=N[C@@H](c2ccccc2)[C@H](c2ccccc2)N1] |
| (S,S)-3b | 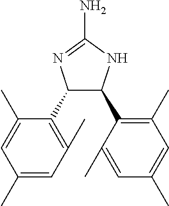 [SMILES: Cc1cc(C)c([C@@H]2N=C(N)N[C@H]2c2c(C)cc(C)cc2C)c(C)c1] |
| 4a | 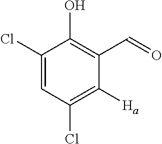 |
| 4b | 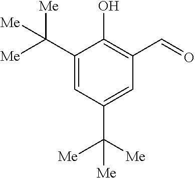 [SMILES: CC(C)(C)c1cc(C=O)c(O)c(C(C)(C)C)c1] |
| 4c | 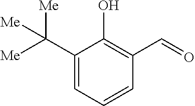 [SMILES: CC(C)(C)c1cccc(C=O)c1O] |
| 5a | 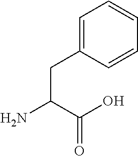 [SMILES: NC(Cc1ccccc1)C(=O)O] |
| 5b | 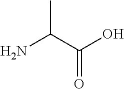 |
| 5c | 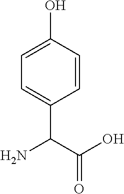 [SMILES: NC(C(=O)O)c1ccc(O)cc1] |
| 5d | 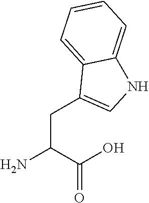 [SMILES: NC(Cc1c[nH]c2ccccc12)C(=O)O] |
| 5e | 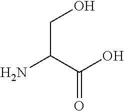 |
| 5f | 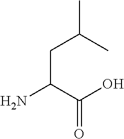 |
| 5g | 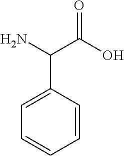 [SMILES: NC(C(=O)O)c1ccccc1] |
| 5h | 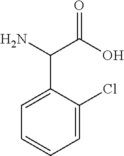 [SMILES: NC(C(=O)O)c1ccccc1Cl] |
| 6a | 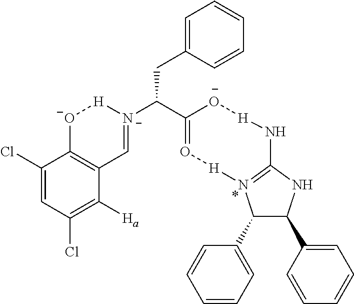 |
| 6b | 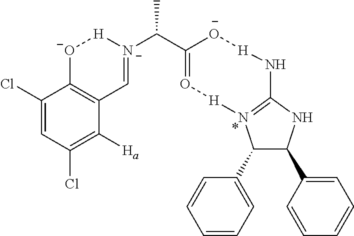 |
| 6c | 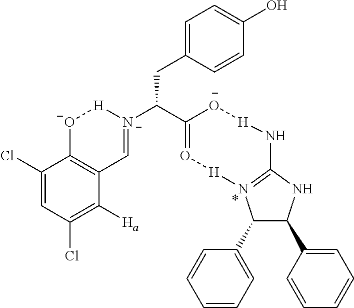 |
| 6d | 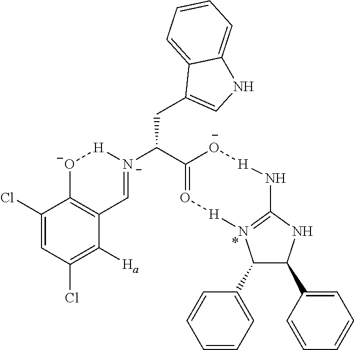 |
| 6e | 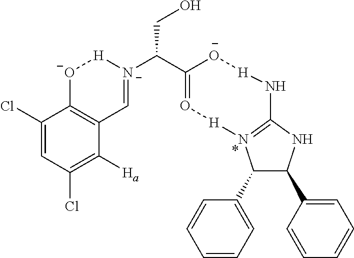 |
| 6f | 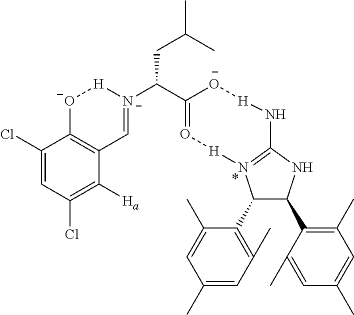 |
| 6g | 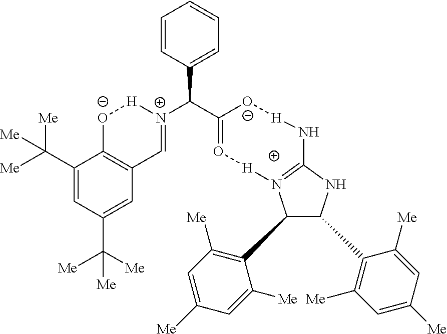 |
| 6h | 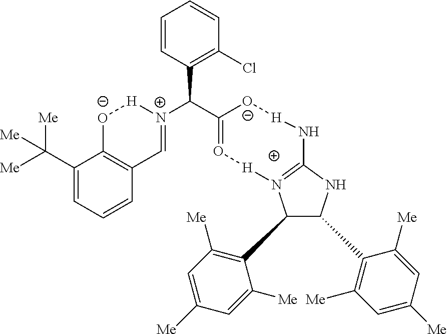 |

A solution of L-phenylalanine, (4a) and (S,S)-3a in acetonitrile was stirred at 40° C. for 5 h (FIG. 1). The clear solution was allowed to cool to room temperature and stirred for another 20 h for SIDT to take place. The precipitated imino acid salt ((S,S)-D-6a) was filtered (83% yield, >99:1 dr). Stirring the mixture for an additional 2 days resulted in 90% yield. Addition of (S,S)-D-6a to acetonitrile with 3% (v/v) conc. HCl resulted in hydrolysis of the imino acid salt and precipitation of the D-amino acid hydrochloride salt (>98% ee). 3 and 4 were recovered by stirring 6 in 0.5 M HCl (precipitation of 4) followed by basification of the filtrate (precipitation of 3).

The ratio of concentrations of (S, S)-D-6a and (S, S)-L-6a can be determined from ¹H NMR by integrating the Hₐ signals (FIG. 1) of the two diastereomeric salts. There are about equal concentrations of (S,S)-L-6a and (S,S)-D-6a in the initial reaction mixture after heating at 40° C. for 5 h (FIG. 2a). After SIDT, the Hₐ signal due to (S,S)-L-6a has essentially disappeared (FIG. 2b). Interestingly, the Hₐ signals for (S,S)-D-6a-e appear downfield of corresponding (S,S)-L-6a-e signals. Furthermore, (S,S)-D-6a-e are less soluble than corresponding (S,S)-L-6a-e. In case of 5f, the solution failed to yield precipitates during attempts at SIDT using (S,S)-3a and 4a. However, using a different guanidine ((S,S)-3b) with 3,5-dichloro-2-hydroxybenzaldehyde, L to D conversion of 5f was achieved. Deracemisation of racemic 5f was achieved with (R,R)-3b, resulting in diastereomerically pure (R,R)-L-6f in 70% yield (Table 1, entry 7).

SIDT was also successful using alpha-arylglycines as substrates (Table 1, entries 8-9). However, due to the increased activation of the alpha-carbon by the alpha-aryl group, decarboxylation was observed when the electron-withdrawing 3,5-dichlorosalicylaldehyde (4a) was used. Upon switching to 3,5-di-tert-butylsalicylaldehyde (4b) and 3-tert-butylsalicylaldehyde (4c), it was possible to obtain the diastereomeric salts of L-phenylglycine (Table 1, entry 8) and L-2-chlorophenylglycine (Table 1, entry 9) from their racemic mixtures in 78% and 73% yields respectively.

It is worth noting that the sense of stereoselectivity remains the same for 3b as for 3a. Thus the Hₐ signal for (S, S)-D-6f is downfield that of (S, S)-L-6f and (S, S)-D-6f is less soluble than (S, S)-L-6f.

TABLE 1
SIDT of imino acid complexes (6)

| | | | | Yield | |
|---|---|---|---|---|---|
| Entry | Substrate | Guanidine | Product | (%)^{[a]} | d.r.^{[b]} |
| 1 | L-5a | (S, S)-3a | (S, S)-D-6a | 83 | >99:1 |
| 2 | L-5b | (S, S)-3a | (S, S)-D-6b | 64 | >99:1 |
| 3 | L-5c | (S, S)-3a | (S, S)-D-6c | 92 | >99:1 |
| 4 | L-5d | (S, S)-3a | (S, S)-D-6d | 91 | 98:2 |
| 5 | L-5e | (S, S)-3a | (S, S)-D-6e | 78 | >99:1 |
| 6 | L-5f | (S, S)-3b | (S, S)-D-6f | 71 | >99:1 |
| 7 | DL-5f | (R, R)-3b | (R, R)-L-6f | 70 | >99:1 |
| 8 | DL-5g | (R, R)-3b | (R, R)-L-6g | 78 | >99:1 |
| 9 | DL-5h | (R, R)-3b | (R, R)-L-6h | 73 | 97:3 |

^{[a]}Isolated yield of imino acid complex.
^{[b]}d.r. values were determined by ¹H NMR analysis of isolated imino acid complexes.

In addition to the Hₐ signal, other ¹H NMR signals can be used to determine the ratio of concentrations of (S,S)-D-6a-f and (S,S)-L-6a-f. There is excellent agreement between the concentration ratios obtained from integration of Hₐ signals and other ¹H NMR signals. Thus, this system allows for simultaneous deracemisation and determination of diastereomeric purity.

One of the main conditions for a successful SIDT is that racemisation be faster than crystallization. There are two special types of strong hydrogen bonds in this system (Scheme 2) that appear to play important roles in the rapid racemisation. The first is the resonance-assisted hydrogen bond (RAHB) between the phenolic oxygen and the imine nitrogen and the second is the charged double-hydrogen bond between the carboxylate and the guanidinium groups. Insight into the role of the hydrogen bonds in speeding up the racemisation may be obtained from the crystallographic and computational studies discussed below.

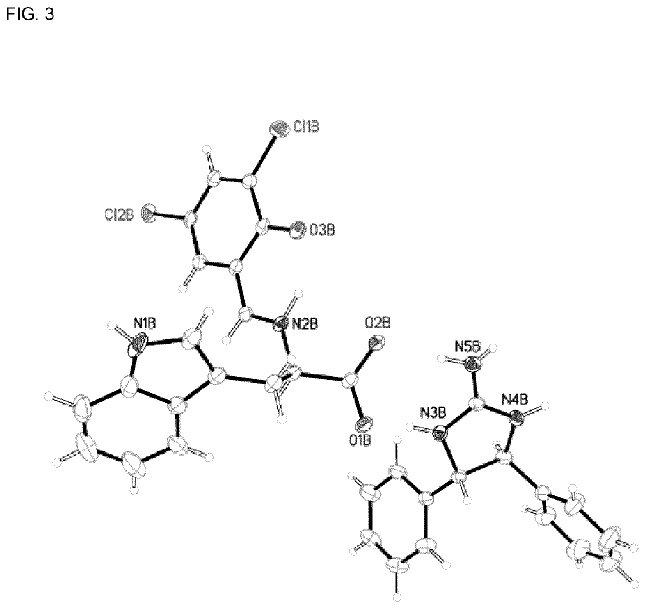

FIG. 3 shows the crystal structure of (S,S)-D-6d. The hydrogen involved in the RAHB is attached to the imine nitrogen and hydrogen bonded to the phenolic oxygen. Computation also shows that (S,S)-D-6d is more stable when the proton is attached to the imine nitrogen than when it is attached to the phenolic oxygen. If the two chloro-substituents in (S,S)-D-6d are removed, computation then shows that the hydrogen is favored to be on the phenolic oxygen rather than the imine nitrogen. The phenolic group in (S,S)-D-6d is sufficiently electron deficient for the proton to prefer the imine nitrogen over the phenolic oxygen. This is consistent with the NMR studies as the proton is observed to be significantly more downfield (15 ppm) than in other compounds when the proton is on the phenolic oxygen (13 ppm). The positive charge on the imine nitrogen in (S,S)-D-6d may acidify the alpha-proton of the imino acid. This is corroborated by the observation that deuteration of the alpha-carbon of phenylalanine occurs more rapidly with dichlorosalicylaldehyde (4) than with salicylaldehyde.

### Example 10

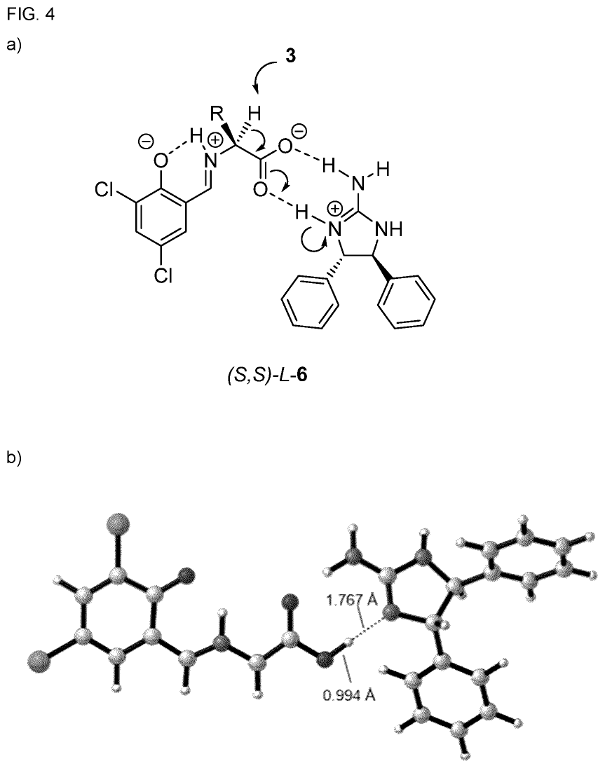

In order to gain more insight into the rapid racemisation, we carried out DFT computation of the imino acid ion pair complex (6) at the B3LYP/6-31 G* level of theory. In general, base catalysts are needed for racemisation of amino acids. Neutral guanidine (3) can play this role. However, computation indicates that there may be an additional general acid catalysis coming from the guanidinium group in the ion pair complex (FIG. 4). The carboxylate group in the ion pair complex is not basic enough to deprotonate the guanidinium counter ion. However, computation reveals that once the alpha-proton is removed with a base, the carboxylate group becomes basic enough to deprotonate the guanidinium. Computation of the complex was started as the carboxylate guanidinium salt. After minimisation, the proton has transferred from the guanidinium nitrogen to the carboxylate oxygen. The distance between the proton and nitrogen is 1.767 angstroms while the distance between the proton and oxygen is 0.994 angstroms (FIG. 4b). Thus, removal of proton from the alpha-position is expected to be concerted with proton transfer from the guanidinium to the carboxylate group in the ion pair complex. This is in accordance with Jencks' “libido-rule” and matching-pKₐ requirement for general acid/base catalysis. Pre-association between the carboxyl group and the guanidium group by charge assisted hydrogen bonding would allow efficient proton transfer at the transition state resulting in general acid catalysis. Racemisation is more rapid with this system in less polar solvents like acetonitrile where tight ion pairs would form. This is in contrast to other systems where racemisation is faster in more polar solvents like water or alcohol.

### Example 11: Tryptophan

To a solution of 3,5-dichlorosalicylaldehyde (11 mmol, 2.1 g) and (S,S)-DPEN guanidine (15 mmol, 3.56 g) dissolved in acetonitrile (50 mL) was added L-tryptophan (10 mmol, 2.04 g) at 60° C. After stirring for 5 hours at 60° C., the reaction mixture was cooled to room temperature and stirred for 20 hours. The precipitates were filtered and redissolved in isopropanol (50 mL) and stirred at 40° C. for 12 hours. After, the reaction mixture was cooled to room temperature and stirred at room temperature for 2 hours. The yellow precipitates were filtered and dried under vacuum. (91% yield).

Hydrolysis of imine salt: To a solution of BnNH₃Cl (1.79 mmol) in isopropanol (16 mL)/H₂O (1 mL) was added imine salt (1.63 mmol). The solution was stirred for 1 hour at room temperature and filtered under vacuum. A solution filtered solid in dichloromethane was stirred for 1 hour at room temperature and was then filtered and dried under vacuum. (70% yield).

### Example 12: Tyrosine

To a solution of 3,5-dichlorosalicylaldehyde (11 mmol, 2.1 g) and (S,S)-DPEN guanidine (15 mmol, 3.56 g) dissolved in methanol (50 mL) was added L-tyrosine (10 mmol, 1.81 g) at 60° C. After stirring for 5 hours at 60° C., methanol was evaporated and acetonitrile (50 mL) was added. The reaction mixture was stirred at 40° C. for 20 hours followed by stirring at room temperature for 2 hours. The yellow precipitates were then filtered by vacuum and dried under vacuum. (92% yield)

Hydrolysis of imine salt: To a solution of concentrated hydrochloric acid (3 mmol) in acetonitrile (52 mL) was added imine salt (1 mmol). The solution was stirred for 1 hour at room temperature and filtered under vacuum. To a solution of filtered solid in H₂O (10 mL) was stirred for 10 minutes and was slowly added trimethylamine (1.1. mmol) at room temperature. After 1 hour, the reaction mixture was filtered and dried under vacuum. (90% yield)

### Example 13: Serine

To a solution of 3,5-dichlorosalicylaldehyde (11 mol, 2.1 g) and (S,S)-DPEN guanidine (15 mmol, 3.56 g) dissolved methanol (50 mL) was added L-serine (10 mmol, 1.05 g) at 40° C. After stirring for 5 hours at 50° C., methanol was evaporated and acetonitrile (50 mL) was added. The reaction mixture was stirred for 3 hours at 40° C. followed by stirring at room temperature of 20 hours. The yellow precipitates were filtered and dried under vacuum. A solution of filtered solids in dichloromethane (50 mL) was stirred for 2 hours at room temperature and was filtered and dried under vacuum. (78%) yield.

Hydrolysis of imine salt: To a solution of concentrated hydrochloric acid (3 mmol) in acetonitrile (10 mL) was added imine salt. The solution was stirred for 1 hour at room temperature and filtered under vacuum. To a solution of filtered solid (1 mmol) in dichloromethane (5 mL)/95% ethanol (5 mL) was stirred for 10 minutes and was slowly added trimethylamine (1.1 mmol) at room temperature. After 1 hour, the reaction mixture was filtered and dried under vacuum. (85% yield)

### Example 14: Alanine

3,5-dichlorosalicylaldehyde (0.0525 g, 0.253 mmol) and (S,S)-diphenyl guanidine (0.0890 g, 0.375 mmol) was dissolved in methanol (1.25 mL). L-alanine (0.0223 g, 0.5 mmol) was added to the solution and stirred at 40° C. for 4 hours. Then the methanol was evaporated and acetonitrile (1.25 mL) was added and the solution was stirred at 40° C. for 2 hours and was then cooled to room temperature and stirred for 19 hours. The yellow precipitates were then filtered and washed with cold acetonitrile and hexanes and dried under vacuum. (64% yield)

### Example 15: Leucine

3,5-dichlorosalicylaldehyde (0.0210 g, 0.11 mmol) and mesityl guanidine (0.0482 g, 0.15 mmol) was dissolved in methanol (0.5 mL). Leucine (0.0131 g, 0.1 mmol) was added to the solution and stirred at 50° C. for 4 hours. Then the methanol was evaporated and acetonitrile (0.5 mL) was added and continued stirring at 50° C. for 2 hours. The solution was allowed to cool to room temperature and stirred at room temperature for 20 hours. Yellow precipitates crashed out of solution and were filtered and washed with cold acetonitrile and hexanes and dried under vacuum. (71% yield with (S,S), 70% yield with (R,R))

### Example 16: Phenylalanine

To solution of 3,5-dichlorosalicylaldehyde (11 mmol, 2.1 g) and (S,S)-DPEN guanidine (15 mmol, 3.56 g) dissolved in acetonitrile (50 mL) was added L-phenylalanine (10 mmol, 1.65 g) at 40° C. After stirring for 5 hours at 40° C., the reaction mixture was cooled to room temperature and stirred for 20 hours. The yellow precipitates were then filtered and dried under vacuum (83% yield).

To a solution of concentrated hydrochloric acid (15.6 mmol) in acetonitrile (52 mL) was added imine salt (5.2 mmol). The solution was stirred for 1 hour at room temperature and filtered under vacuum. A solution of filtered solid in 1:1 DCM/95% ethanol (50 mL) was stirred for 10 minutes and was slowly added triethylamine (5.7 mmol) at room temperature. After 1 hour, the reaction mixture was filtered and dried under vacuum. (85% yield)

### Example 17: Phenylglycine

3,5-Di-tert-butylsalicylaldehyde (0.11 mmol, 0.0258 g) and (R,R)-mesityl guanidine (0.15 mmol, 0.0482 g) was dissolved in methanol (0.5 mL). DL-Phenylglycine (0.1 mmol, 0.0151 g) was added to the solution and stirred at 50° C. for 30 minutes. Then methanol was evaporated and acetonitrile (0.5 mL) was added. Solution was stirred at room temperature for 70 hours. Precipitates were filtered and washed with cold acetonitrile and dried under vacuum. (78% yield)

### Example 18: 2-Chlorophenylglycine

3-Tert-butylsalicylaldehyde (0.11 mmol, 0.019 mL) and (R,R)-mesityl guanidine (0.15 mmol, 0.0482 g) was dissolved in methanol (0.5 mL). DL-2-Chlorophenylglycine (0.1 mmol, 0.0185 g) was added to the solution and stirred at 50° C. for 30 minutes. Then methanol was evaporated and acetonitrile was added. Solution was stirred at room temperature for 22 hours. Precipitates were filtered and washed with cold acetonitrile and dried under vacuum. (73% yield)

### Example 19: Solubility-Induced Diastereomer Transformation (SIDT) of Tryptophan

1. L to D Conversion Starting from L-Tryptophan

SIDT: To solution of 3,5-dichlorosalicylaldehyde (1.1 equiv.) and (S,S)-diphenylethylene diamine guanidine (1.5 equiv.) dissolved in acetonitrile (0.2 M) was added L-tryptophan (1.0 equiv.) at 60° C. (aldehyde and guanidine are not completely soluble in acetonitrile and only fully dissolves after addition of amino acid and formation of imine salt). After stirring for 5 hours at 60° C., the reaction mixture was cooled to room temperature and stirred for 20 hours to induce precipitation. The yellow precipitates were filtered and dissolved in isopropanol (50 mL) and stirred at 40° C. for 12 hours. After, the reaction mixture was cooled to room temperature and stirred at room temperature for 2 hours. The yellow precipitates were filtered and dried under vacuum.

Hydrolysis of imine salt: To a solution of BnNH₃Cl (1.1 equiv.) in 16:1 isopropanol/H₂O (1 M) was added imine salt (1.0 equiv.). The solution was stirred for 1 hour at room temperature and white precipitates were filtered under vacuum.

2. D to L Conversion Starting from D-Tryptophan

SIDT: To solution of 3,5-dichlorosalicylaldehyde (1.1 equiv.) and (R,R)-diphenylethylene diamine guanidine (1.5 equiv.) dissolved in acetonitrile (0.2 M) was added D-tryptophan (1.0 equiv.) at 60° C. (aldehyde and guanidine are not completely soluble in acetonitrile and only fully dissolves after addition of amino acid and formation of imine salt). After stirring for 5 hours at 60° C., the reaction mixture was cooled to room temperature and stirred for 20 hours to induce precipitation. The yellow precipitates were filtered and dissolved in isopropanol (50 mL) and stirred at 40° C. for 12 hours. After, the reaction mixture was cooled to room temperature and stirred at room temperature for 2 hours. The yellow precipitates were filtered and dried under vacuum.

Hydrolysis of imine salt: To a solution of BnNH₃Cl (1.1 equiv.) in 16:1 isopropanol/H₂O (1 M) was added imine salt (1.0 equiv.). The solution was stirred for 1 hour at room temperature and white precipitates were filtered under vacuum.

3. L-Tryptophan from Deracemisation of Racemic (DL) Tryptophan

SIDT: To solution of 3,5-dichlorosalicylaldehyde (1.1 equiv.) and (S,S)-diphenylethylene diamine guanidine (1.5 equiv.) dissolved in acetonitrile (0.2 M) was added DL-tryptophan (1.0 equiv.) at 60° C. (aldehyde and guanidine are not completely soluble in acetonitrile and only fully dissolves after addition of amino acid and formation of imine salt). After stirring for 5 hours at 60° C., the reaction mixture was cooled to room temperature and stirred for 20 hours to induce precipitation. The yellow precipitates were filtered and dissolved in isopropanol (50 mL) and stirred at 40° C. for 12 hours. After, the reaction mixture was cooled to room temperature and stirred at room temperature for 2 hours. The yellow precipitates were filtered and dried under vacuum.

Hydrolysis of imine salt: To a solution of BnNH₃Cl (1.1 equiv.) in 16:1 isopropanol/H₂O (1 M) was added imine salt (1.0 equiv.). The solution was stirred for 1 hour at room temperature and white precipitates were filtered under vacuum.

4. D-Tryptophan from Deracemisation of Racemic (DL) Tryptophan

SIDT: To solution of 3,5-dichlorosalicylaldehyde (1.1 equiv.) and (R,R)-diphenylethylene diamine guanidine (1.5 equiv.) dissolved in acetonitrile (0.2 M) was added DL-tryptophan (1.0 equiv.) at 60° C. (aldehyde and guanidine are not completely soluble in acetonitrile and only fully dissolves after addition of amino acid and formation of imine salt). After stirring for 5 hours at 60° C., the reaction mixture was cooled to room temperature and stirred for 20 hours to induce precipitation. The yellow precipitates were filtered and dissolved in isopropanol (50 mL) and stirred at 40° C. for 12 hours. After, the reaction mixture was cooled to room temperature and stirred at room temperature for 2 hours. The yellow precipitates were filtered and dried under vacuum.

Hydrolysis of imine salt: To a solution of BnNH₃Cl (1.1 equiv.) in 16:1 isopropanol/H₂O (1 M) was added imine salt (1.0 equiv.). The solution was stirred for 1 hour at room temperature and white precipitates were filtered under vacuum.

### 5. Determination of Enantiopurity of Tryptophan

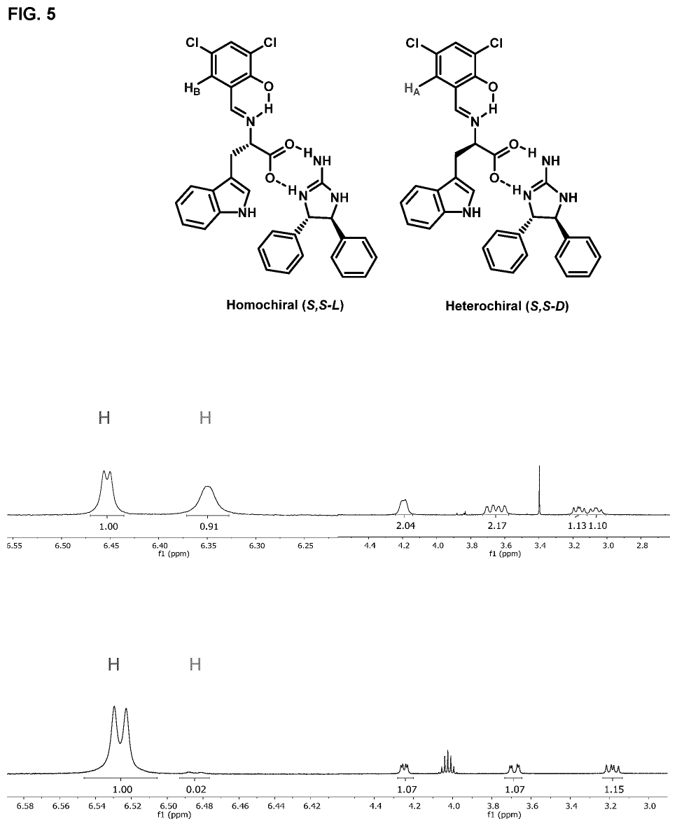

Sample of tryptophan of interest (1.0 equiv.) is dissolved in solution of 3,5-dichlorosalicylaldehyde (1.1 equiv.) and diphenylethylene guanidine (1.0 equiv.) A small excess of aldehyde over guanidine in solution was used to prevent in situ epimerization. The Hₐ belonging to the heterochiral complex ((S,S)-D or (R,R)-L) is more downfield than the Hₐ belonging to the homochiral complex ((S,S)-L or (R,R)-D). And the integration between the two signals will give the ratio of the two enantiomers of tryptophan (see FIG. 5).

### Example 20: Solubility-Induced Diastereomer Transformation (SIDT) of 2-Chlorophenylglycine

1. L to D Conversion Starting from L-2-Chlorophenylglycine

SIDT: 3-tert-butylsalicylaldehyde (1.1 equiv.) and (S,S)-mesityl guanidine (1.5 equiv.) was partially dissolved in methanol (0.2 M). L-2-Chlorophenylglycine (1.0 equiv.) was added to the solution and stirred at 50° C. for 30 minutes. Then methanol was evaporated and acetonitrile was added (imine guanidinium salt is soluble in methanol but not acetonitrile). Solution was stirred at room temperature for 20 hours (precipitation occurred within the first 10 minutes of stirring in acetonitrile). Beige precipitates were filtered and washed with cold acetonitrile and dried under vacuum.

Hydrolysis of imine salt: To a solution of concentrated HCl (3.0 equiv.) in acetonitrile (0.1 M) was added imine salt (1.0 equiv.). The solution was stirred for 1 hour at room temperature and white precipitates were filtered under vacuum. A solution of filtered solid in 1:1 DCM/95% ethanol (0.1 mL) was stirred for 10 minutes and was slowly added triethylamine (1.1 equiv.) at room temperature. After 1 hour, the reaction mixture was filtered and dried under vacuum.

2. D to L Conversion Starting from D-2-Chlorophenylglycine

SIDT: 3-tert-butylsalicylaldehyde (1.1 equiv.) and (R,R)-mesityl guanidine (1.5 equiv.) was partially dissolved in methanol (0.2 M). D-2-Chlorophenylglycine (1.0 equiv.) was added to the solution and stirred at 50° C. for 30 minutes. Then methanol was evaporated and acetonitrile was added (imine guanidinium salt is soluble in methanol but not acetonitrile). Solution was stirred at room temperature for 20 hours (precipitation occurred within the first 10 minutes of stirring in acetonitrile). Beige precipitates were filtered and washed with cold acetonitrile and dried under vacuum.

Hydrolysis of imine salt: To a solution of concentrated HCl (3.0 equiv.) in acetonitrile (0.1 M) was added imine salt (1.0 equiv.). The solution was stirred for 1 hour at room temperature and white precipitates were filtered under vacuum. A solution of filtered solid in 1:1 DCM/95% ethanol (0.1 mL) was stirred for 10 minutes and was slowly added triethylamine (1.1 equiv.) at room temperature. After 1 hour, the reaction mixture was filtered and dried under vacuum.

3. L-2-Chlorophenylglycine from Deracemisation of Racemic (DL) 2-Chlorophenylglycine

SIDT: 3-tert-butylsalicylaldehyde (1.1 equiv.) and (R,R)-mesityl guanidine (1.5 equiv.) was partially dissolved in methanol (0.2 M). DL-2-Chlorophenylglycine (1.0 equiv.) was added to the solution and stirred at 50° C. for 30 minutes. Then methanol was evaporated and acetonitrile was added (imine guanidinium salt is soluble in methanol but not acetonitrile). Solution was stirred at room temperature for 20 hours (precipitation occurred within the first 10 minutes of stirring in acetonitrile). Beige precipitates were filtered and washed with cold acetonitrile and dried under vacuum.

Hydrolysis of imine salt: To a solution of concentrated HCl (3.0 equiv.) in acetonitrile (0.1 M) was added imine salt (1.0 equiv.). The solution was stirred for 1 hour at room temperature and white precipitates were filtered under vacuum. A solution of filtered solid in 1:1 DCM/95% ethanol (0.1 mL) was stirred for 10 minutes and was slowly added triethylamine (1.1 equiv.) at room temperature. After 1 hour, the reaction mixture was filtered and dried under vacuum.

4. D-2-Chlorophenylglycine from Deracemisation of Racemic (DL) 2-Chlorophenylglycine

SIDT: 3-tert-butylsalicylaldehyde (1.1 equiv.) and (S,S)-mesityl guanidine (1.5 equiv.) was partially dissolved in methanol (0.2 M). DL-2-Chlorophenylglycine (1.0 equiv.) was added to the solution and stirred at 50° C. for 30 minutes. Then methanol was evaporated and acetonitrile was added (imine guanidinium salt is soluble in methanol but not acetonitrile). Solution was stirred at room temperature for 20 hours (precipitation occurred within the first 10 minutes of stirring in acetonitrile). Beige precipitates were filtered and washed with cold acetonitrile and dried under vacuum.

Hydrolysis of imine salt: To a solution of concentrated HCl (3.0 equiv.) in acetonitrile (0.1 M) was added imine salt (1.0 equiv.). The solution was stirred for 1 hour at room temperature and white precipitates were filtered under vacuum. A solution of filtered solid in 1:1 DCM/95% ethanol (0.1 mL) was stirred for 10 minutes and was slowly added triethylamine (1.1 equiv.) at room temperature. After 1 hour, the reaction mixture was filtered and dried under vacuum.

### 5. Determination of Enantiopurity of 2-Chlorophenylglycine

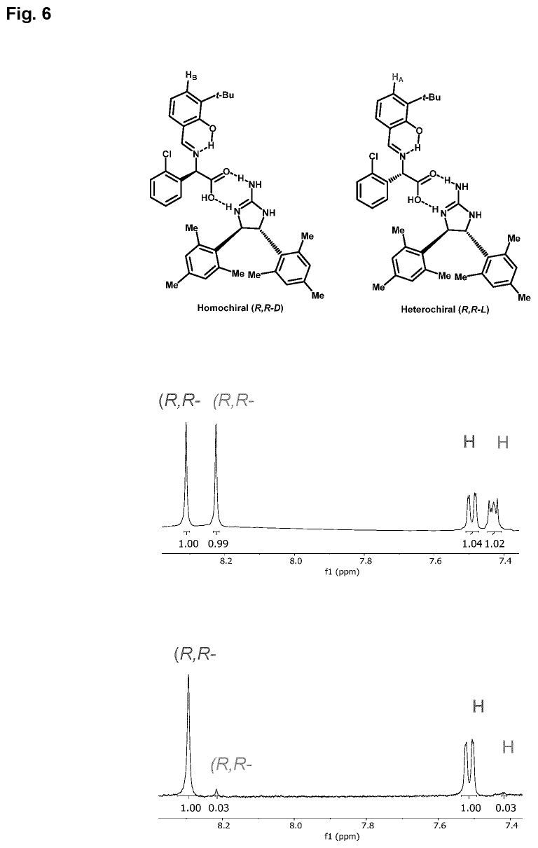

Sample of 2-chlorophenylglycine of interest (1.0 equiv.) is dissolved in solution of 3-tert-butylsalicylaldehyde (1.1 equiv.) and mesityl guanidine (1.0 equiv.) A small excess of aldehyde over guanidine in solution was used to prevent in situ epimerization. The Hₐ belonging to the heterochiral complex ((S,S)-D or (R,R)-L) is more downfield than the H_{b} belonging to the homochiral complex ((S,S)-L or (R,R)-D). And the integration between the two signals will give the ratio of the two enantiomers of tryptophan (see FIG. 6).

### Example 21: Synthesized Imino Acid Guandinium Salts

Procedure: To solution of salicylaldehyde derivative (1.1 equiv.) and chiral guanidine derivative (1.5 equiv.) dissolved in acetonitrile or methanol (0.2 M) was added amino acid (1.0 equiv.) at 40° C. and stirred for 4 hours. The following compounds were successfully synthesized using this procedure:

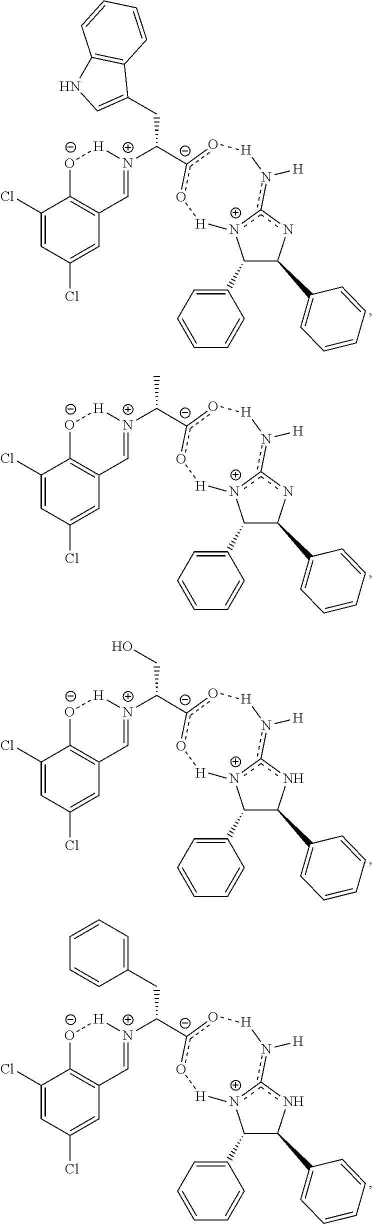

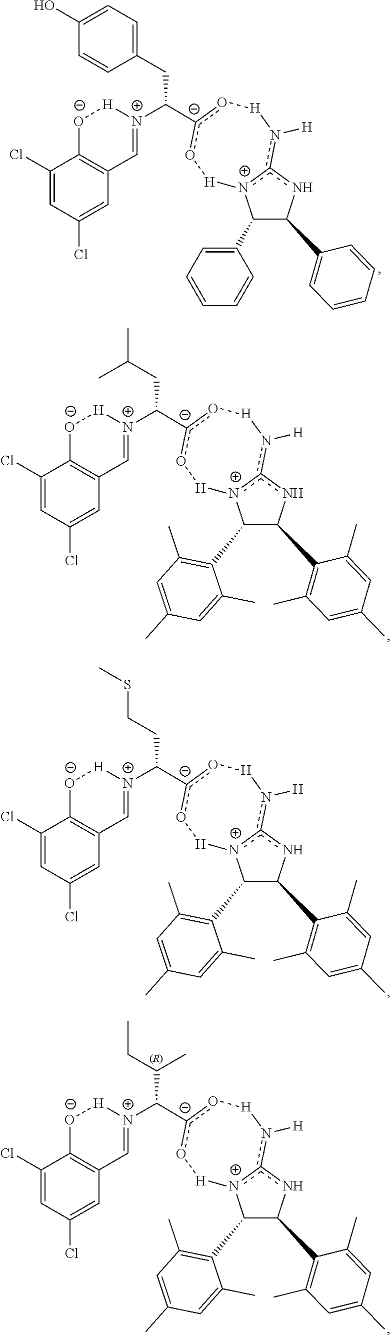

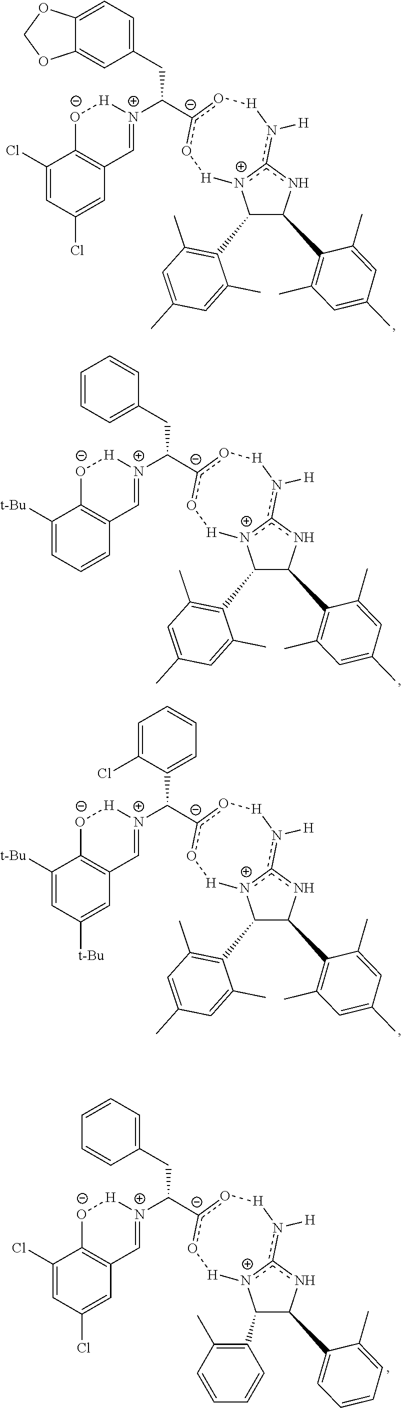

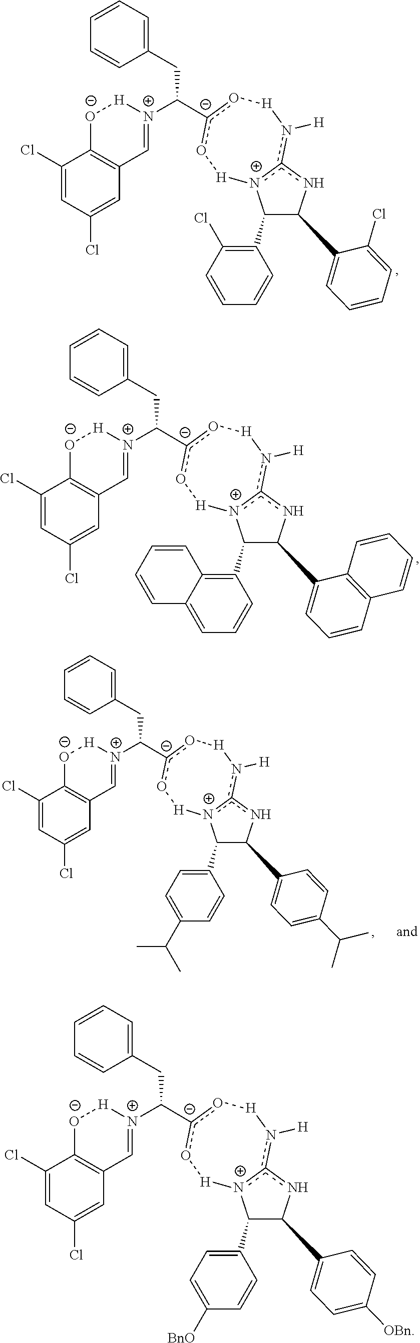

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Furthermore, material appearing in the background section of the specification is not an admission that such material is prior art to the invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. A compound having a structure of Formula (I): 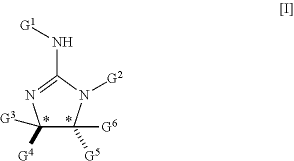
wherein:
both of the chiral carbon atoms denoted by “*” are both in the R configuration or both in the S configuration;
G¹ is selected from the group consisting of: H, C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₆-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)];
G² is selected from the group consisting of: H, C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)];
G³ and G⁶ are independently selected from the group consisting of: H, C₁-C₁₂ substituted alkyl, C₂-C₁₂ unsubstituted alkyl, C₁-C₁₂ substituted heteroalkyl, and C₁-C₁₂ unsubstituted heteroalkyl;
G⁴ and G⁵ are independently selected from the group consisting of: unsubstituted C₆-C₁₂ aryl, substituted C₆-C₁₂ aryl, unsubstituted C₆-C₁₂ heteroaryl, substituted C₆-C₁₂ heteroaryl, unsubstituted [(C₆ aryl)-(C₁-C₆ alkyl)-(C₆ aryl)], substituted [(C₆ aryl)-(C₁-C₆ alkyl)-(C₆ aryl)], unsubstituted [(C₆ aryl)-(C₁-C₆ heteroalkyl)-(C₆ aryl)], and substituted [(C₆ aryl)-(C₁-C₆ heteroalkyl)-(C₆ aryl)];
provided that:
(i) either:
(a) at least one of G¹, G², G³, and G⁶ is not H; or
(b) at least one of G⁴ and G⁵ is not phenyl;
(ii) if G⁴ and G⁶ are both or 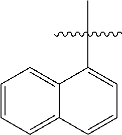 [SMILES: *c1cccc2ccccc12]
or both 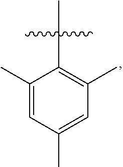
then at least one of G¹, G², G³ and G⁶ is not H;
(iii) if G³ and G⁶ are both H, and G² is methyl, 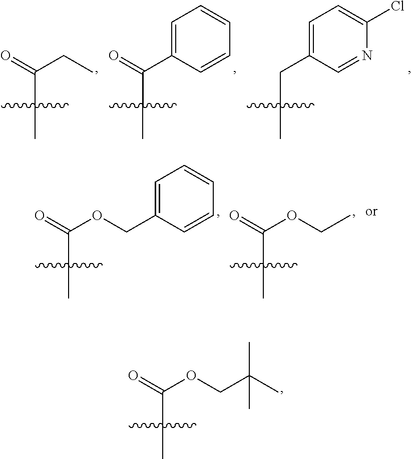
then either G⁴ and G⁵ are not both phenyl, or G¹ is not NO₂, ethyl, tert-butyl, benzyl, cyclohexanyl, 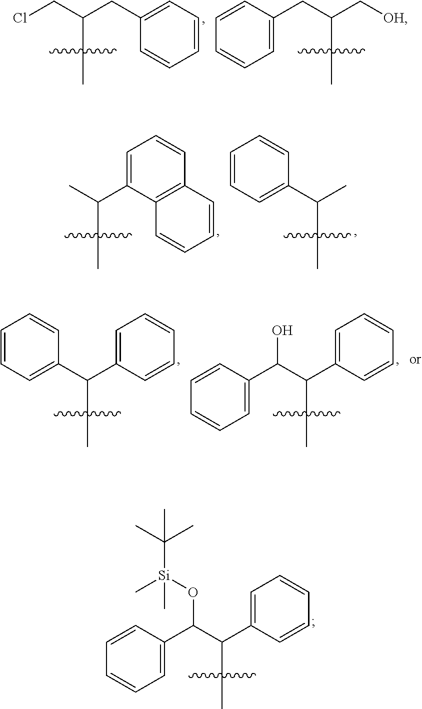
and
(iv) if G², G³, and G⁶ are all H, and G⁴ and G⁵ are both phenyl, then G¹ is not H, methyl, ethyl, tert-butyl, phenyl, benzyl, cyclohexanyl,

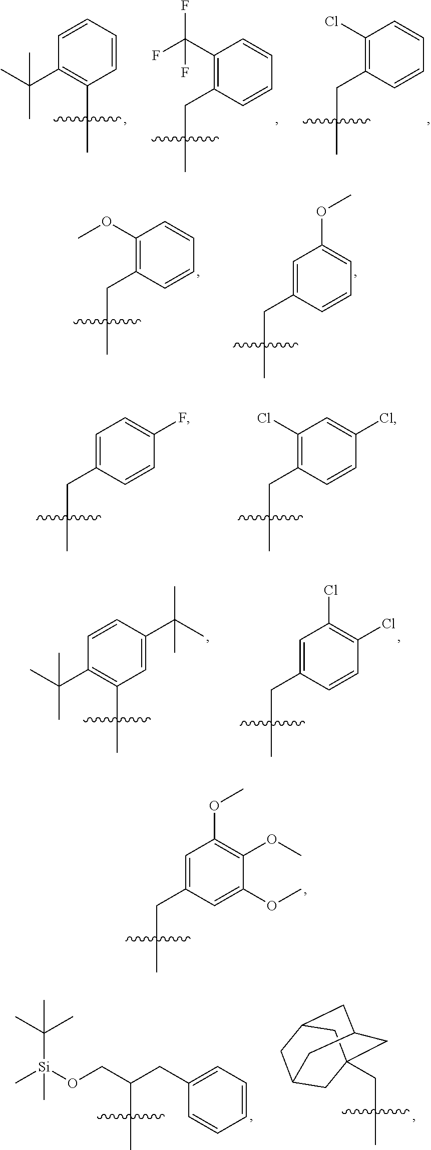

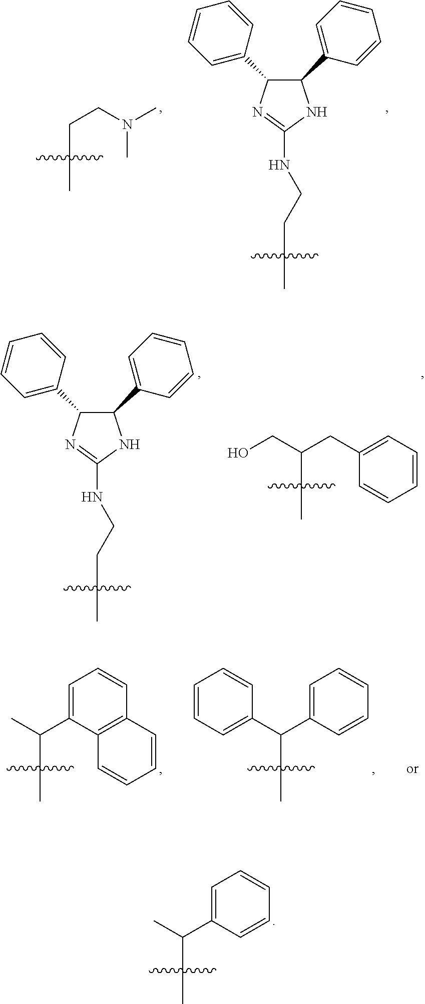

2. The compound of claim 1 wherein if G⁴ and G⁵ are both 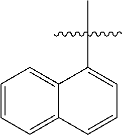 [SMILES: *c1cccc2ccccc12]
or both 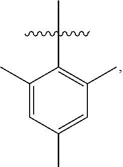
G¹ is not NO₂, phenyl,

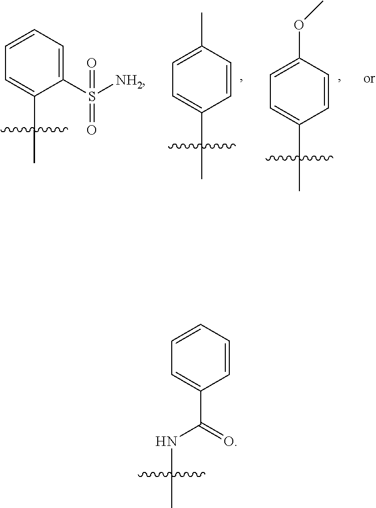

3. The compound of claim 1 wherein if G³ and G⁶ are both H, and G² is methyl, 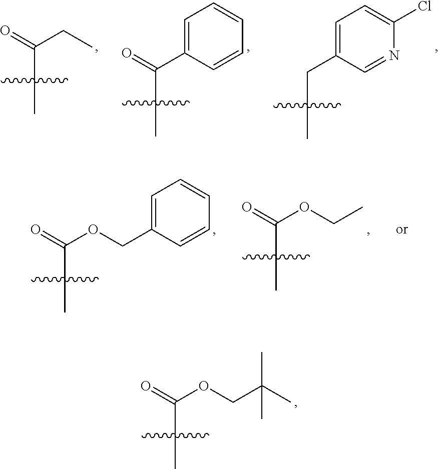
then G¹ is not H.

4. The compound of claim 1 wherein if G², G³, and G⁶ are all H, and G⁴ and G⁵ are both phenyl, then G¹ is not NO₂,

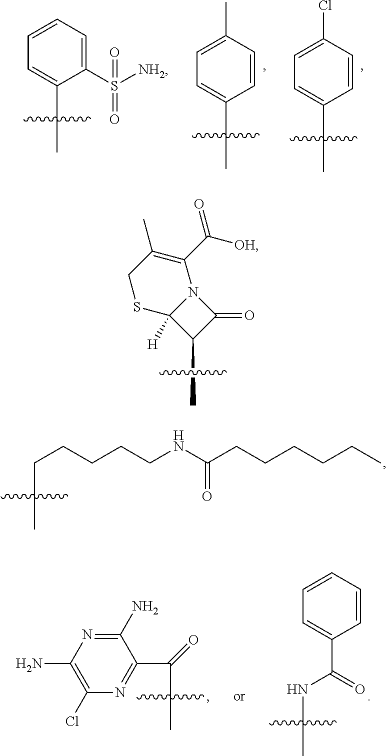

5. The compound of claim 1 wherein the G¹ is not benzyl, substituted benzyl, benzoyl, substituted benzoyl, or —CH₂-cyclohexanyl.

6. The compound of claim 1 wherein the both of the chiral carbon atoms denoted by “*” are both in the S configuration.

7. The compound of claim 1 wherein the both of the chiral carbon atoms denoted by “*” are both in the R configuration.

8. The compound of claim 1 wherein G¹ is selected from the group consisting of: H, C₁-C₆ unsubstituted alkyl, C₁-C₆ substituted alkyl, C₆ unsubstituted aryl, C₆ substituted aryl, unsubstituted (C₁-C₆ alkylene)-(C₆ aryl), substituted (C₁-C₆ alkylene)-(C₆ aryl), C₁-C₆ unsubstituted heteroalkyl, C₁-C₆ substituted heteroalkyl, C₆ unsubstituted heteroaryl, C₆ substituted heteroaryl, unsubstituted hetero[(C₁-C₆ alkylene)-(C₆ aryl)], and substituted hetero[(C₁-C₆ alkylene)-(C₆ aryl)].

9. The compound of claim 1 wherein G² is selected from the group consisting of: H, C₁-C₆ unsubstituted alkyl, C₁-C₆ substituted alkyl, C₆ unsubstituted aryl, C₆ substituted aryl, unsubstituted (C₁-C₆ alkylene)-(C₆ aryl), substituted (C₁-C₆ alkylene)-(C₆ aryl), C₁-C₆ unsubstituted heteroalkyl, C₁-C₆ substituted heteroalkyl, C₆ unsubstituted heteroaryl, C₆ substituted heteroaryl, unsubstituted hetero[(C₁-C₆ alkylene)-(C₆ aryl)], and substituted hetero[(C₁-C₆ alkylene)-(C₆ aryl)].

10. The compound of claim 1 wherein G² is selected from the group consisting of: H, C₁-C₁₂ unsubstituted alkyl, C₁-C₁₂ substituted alkyl, C₆-C₁₂ unsubstituted aryl, C₆-C₁₂ substituted aryl, C₁-C₁₂ unsubstituted heteroalkyl, C₁-C₁₂ substituted heteroalkyl, C₆-C₁₂ unsubstituted heteroaryl, C₆-C₁₂ substituted heteroaryl.

11. The compound of claim 1 wherein G² is selected from the group consisting of: H, C₁-C₆ unsubstituted alkyl, C₁-C₆ substituted alkyl, C₆-C₈ unsubstituted aryl, C₆-C₈ substituted aryl, C₁-C₆ unsubstituted heteroalkyl, C₁-C₆ substituted heteroalkyl, C₅-C₈ unsubstituted heteroaryl, C₅-C₈ substituted heteroaryl.

12. The compound of claim 1 wherein G³ and G⁶ are independently selected from the group consisting of: H, C₁-C₆ substituted alkyl, C₂-C₆ unsubstituted alkyl, C₁-C₆ substituted heteroalkyl, and C₁-C₆ unsubstituted heteroalkyl.

13. The compound of claim 1 wherein G⁴ and G⁵ are independently selected from the group consisting of: unsubstituted C₆ aryl, substituted C₆ aryl, unsubstituted C₆ heteroaryl, and substituted C₆ heteroaryl.

14. The compound of claim 1 wherein the compound has no acid groups.

15. The compound of claim 1 wherein the compound has no charged groups.

16. The compound of claim 1 wherein G³, and G⁶ are both H.

17. The compound of claim 1 wherein G² is H.

18. The compound of claim 1 wherein G⁴ and G⁵ are both a substituted phenyl.

19. The compound of claim 1 wherein G⁴ and G⁵ are phenyl.

20. The compound of claim 1 wherein G¹ is H.

21. A salt having a structure of Formula (II): 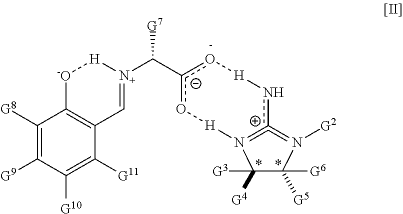
wherein:
both of the chiral carbon atoms denoted by “*” are both in the R configuration or both in the S configuration;
G² is selected from the group consisting of: H, C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)];
G³ and G⁶ are independently selected from the group consisting of: H, C₁-C₁₂ substituted alkyl, C₂-C₁₂ unsubstituted alkyl, C₁-C₁₂ substituted heteroalkyl, and C₁-C₁₂ unsubstituted heteroalkyl;
G⁴ and G⁵ are independently selected from the group consisting of: unsubstituted C₆-C₁₂ aryl, substituted C₆-C₁₂ aryl, unsubstituted C₆-C₁₂ heteroaryl, substituted C₆-C₁₂ heteroaryl, unsubstituted [(C₆ aryl)-(C₁-C₆ alkyl)-(C₆ aryl)], substituted [(C₆ aryl)-(C₁-C₆ alkyl)-(C₆ aryl)], unsubstituted [(C₆ aryl)-(C₁-C₆ heteroalkyl)-(C₆ aryl)], and substituted [(C₆ aryl)-(C₁-C₆ heteroalkyl)-(C₆ aryl)];
G⁷ is selected from the group consisting of: C₁-C₁₂ unsubstituted alkyl, C₁-C₁₂ substituted alkyl, C₆-C₁₂ unsubstituted aryl, C₆-C₁₂ substituted aryl, unsubstituted (C₁-C₆ alkylene)-(C₆-C₁₂ aryl), substituted (C₁-C₆ alkylene)-(C₆-C₁₂ aryl), C₁-C₁₂ unsubstituted heteroalkyl, C₁-C₁₂ substituted heteroalkyl, C₅-C₁₂ unsubstituted heteroaryl, C₅-C₁₂ substituted heteroaryl, unsubstituted hetero[(C₁-C₆ alkylene)-(C₆-C₁₂ aryl)], and substituted hetero[(C₁-C₆ alkylene)-(C₆-C₁₂ aryl)];
and each of G⁸, G⁹, G¹⁰, and G¹¹ are independently selected from the group consisting of: H, halogen, C₁-C₁₂ alkyl, C₆-C₁₂ aryl, C₁-C₁₂ O-alkyl, C₆-C₁₂ O-aryl, C₁-C₁₂ N-dialkyl and C₆-C₁₂ N-diaryl;
provided that the salt of Formula II is other than:

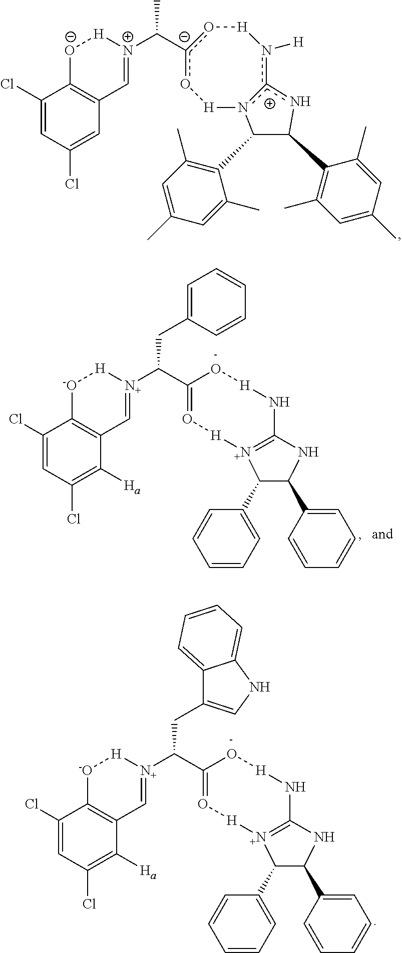

22. The salt of claim 21 wherein G⁷ is a side chain of a natural amino acid or a side chain of an unnatural amino acid.

23. The salt of claim 21 wherein G⁷ is selected from the group consisting of: CH₃, 4-hydroxyphenyl, CH₂OH, propan-2-yl, phenyl, 2-chlorophenyl, CH₂-phenyl, CH₂—CH₂—S—CH₃, butan-2-yl, CH₂-3-H-indole, CH₂-1,3-benxodioxole, CH₂-2,3,4,5,6-pentachloro-phenyl, CH₂-4-nitro-phenyl, CH₂-4-fluoro-phenyl, CH₂-4-bromo-phenyl, CH₂-4-iodo-phenyl, CH₂-cyclohexane, CH₂-naphthyl, CH₂-cylopentane, CH₂-ethynyl, CH₂—C(═CH₂)(CH₃), CH₂-3-H-pyrrole, CH₂—CH₂-phenyl, CH₂-3-H,7-hydroxy-indole, CH₂-3-H,6-hydroxy-indole, and CH2-4-azido-phenyl.

24. The salt of claim 1 wherein G⁸, G⁹, G¹⁰, and G¹¹ are independently selected from the group consisting of: H, halogen, C₁-C₁₂ and unsubstituted alkyl.

25. The salt of claim 1 wherein G⁸ is selected from the group consisting of: H, chloro, and tert-butyl.

26. The salt of claim 1 wherein G⁹ is H.

27. The salt of claim 1 wherein G¹⁰ is selected from the group consisting of: H, chloro, and tert-butyl.

28. The salt of claim 1 wherein G¹¹ is H.

29. The salt of claim 1 wherein the both of the chiral carbon atoms denoted by “*” are both in the S configuration.

30. The salt of claim 1 wherein the both of the chiral carbon atoms denoted by “*” are both in the R configuration.

31. The salt of claim 1 wherein G² is selected from the group consisting of: H, C₁-C₆ unsubstituted alkyl, C₁-C₆ substituted alkyl, C₆ unsubstituted aryl, C₆ substituted aryl, unsubstituted (C₁-C₆ alkylene)-(C₆ aryl), substituted (C₁-C₆ alkylene)-(C₆ aryl), C₁-C₆ unsubstituted heteroalkyl, C₁-C₆ substituted heteroalkyl, C₆ unsubstituted heteroaryl, C₆ substituted heteroaryl, unsubstituted hetero[(C₁-C₆ alkylene)-(C₆ aryl)], and substituted hetero[(C₁-C₆ alkylene)-(C₆ aryl)].

32. The salt of claim 1 wherein G² is selected from the group consisting of: H, C₁-C₁₂ unsubstituted alkyl, C₁-C₁₂ substituted alkyl, C₆-C₁₂ unsubstituted aryl, C₆-C₁₂ substituted aryl, C₁-C₁₂ unsubstituted heteroalkyl, C₁-C₁₂ substituted heteroalkyl, C₆-C₁₂ unsubstituted heteroaryl, C₆-C₁₂ substituted heteroaryl.

33. The salt of claim 1 wherein G² is selected from the group consisting of: H, C₁-C₆ unsubstituted alkyl, C₁-C₆ substituted alkyl, C₆-C₈ unsubstituted aryl, C₆-C₈ substituted aryl, C₁-C₆ unsubstituted heteroalkyl, C₁-C₆ substituted heteroalkyl, C₆-C₈ unsubstituted heteroaryl, C₅-C₈ substituted heteroaryl.

34. The salt of claim 1 wherein G² is H.

35. The salt of claim 1 wherein G³ and G⁶ are independently selected from the group consisting of: H, C₁-C₆ substituted alkyl, C₂-C₆ unsubstituted alkyl, C₁-C₆ substituted heteroalkyl, and C₁-C₆ unsubstituted heteroalkyl.

36. The salt of claim 1 wherein G³, and G⁶ are both H.

37. The salt of claim 1 wherein G⁴ and G⁵ are independently selected from the group consisting of: unsubstituted C₆ aryl, substituted C₆ aryl, unsubstituted C₆ heteroaryl, and substituted C₆ heteroaryl.

38. The salt of claim 1 wherein G⁴ and G⁵ are both a substituted phenyl.

39. The salt of claim 1 wherein G⁴ and G⁵ are phenyl.

40. The salt of claim 1 wherein G⁴ and G⁵ are 2,4,6-trimethyl-phenyl.

41. The salt of claim 21 selected from the group consisting of: 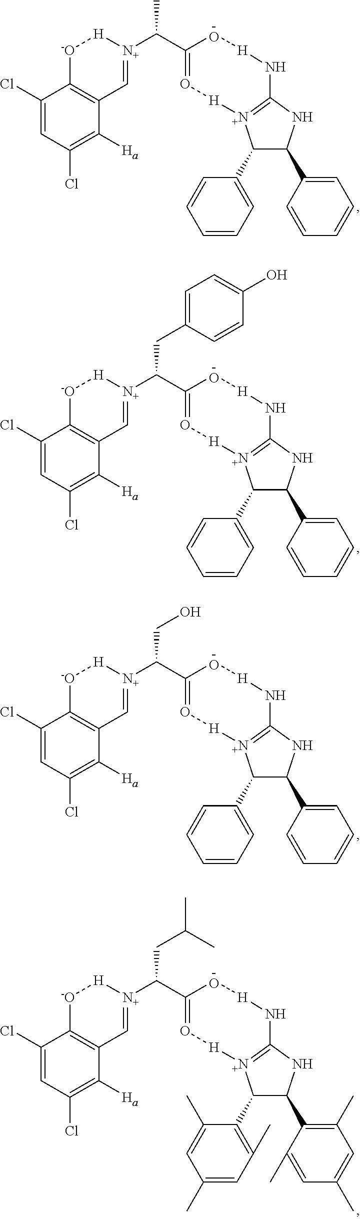 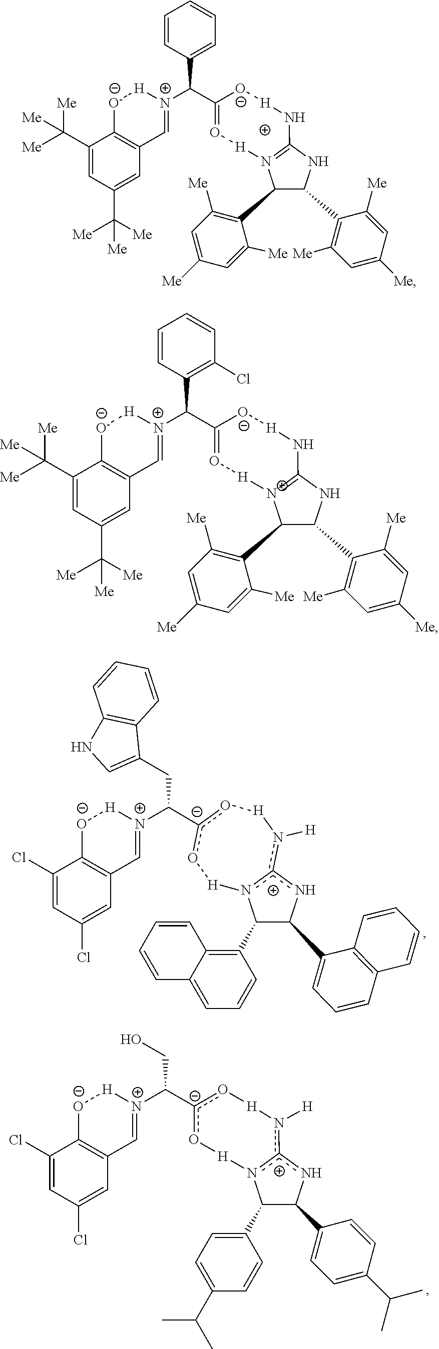 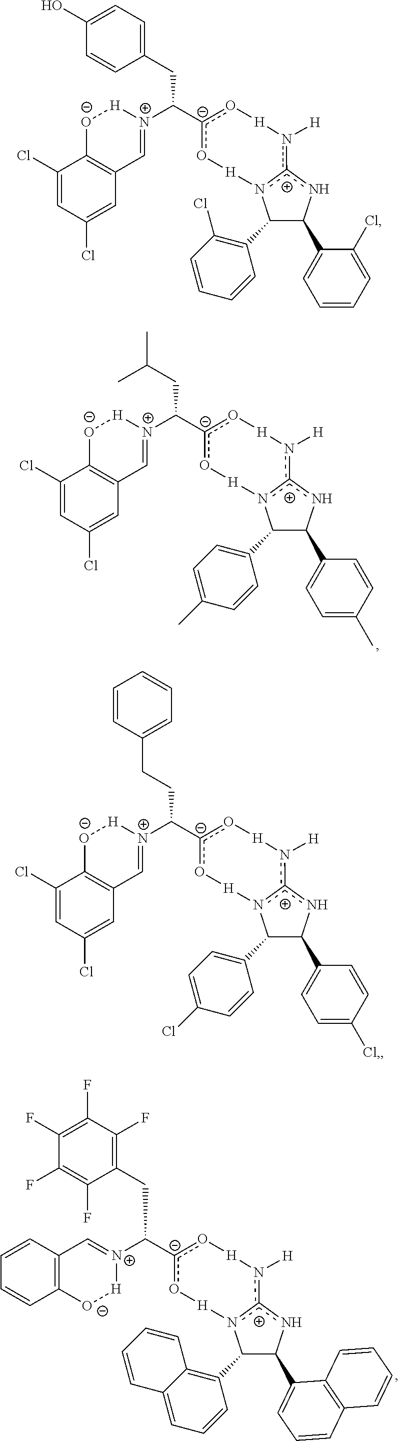 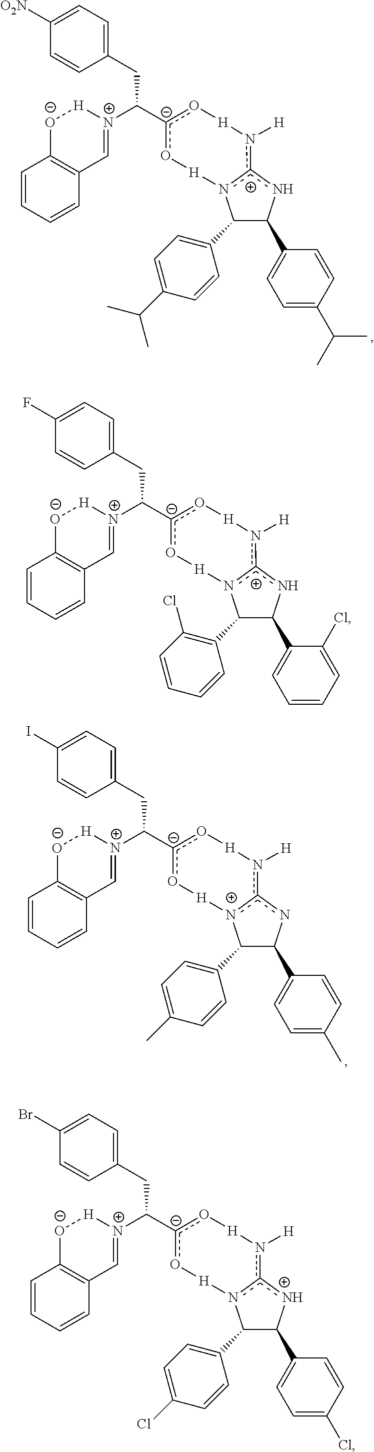 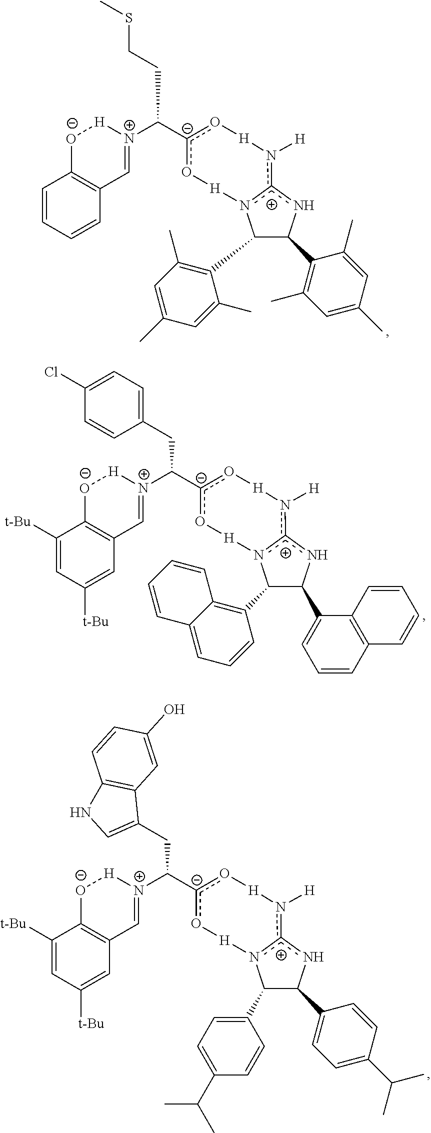 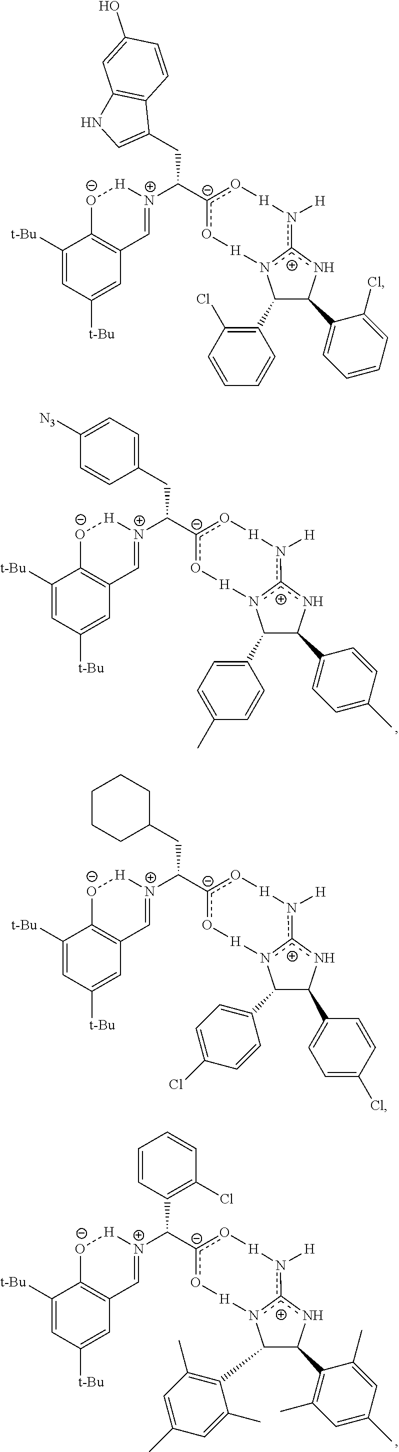 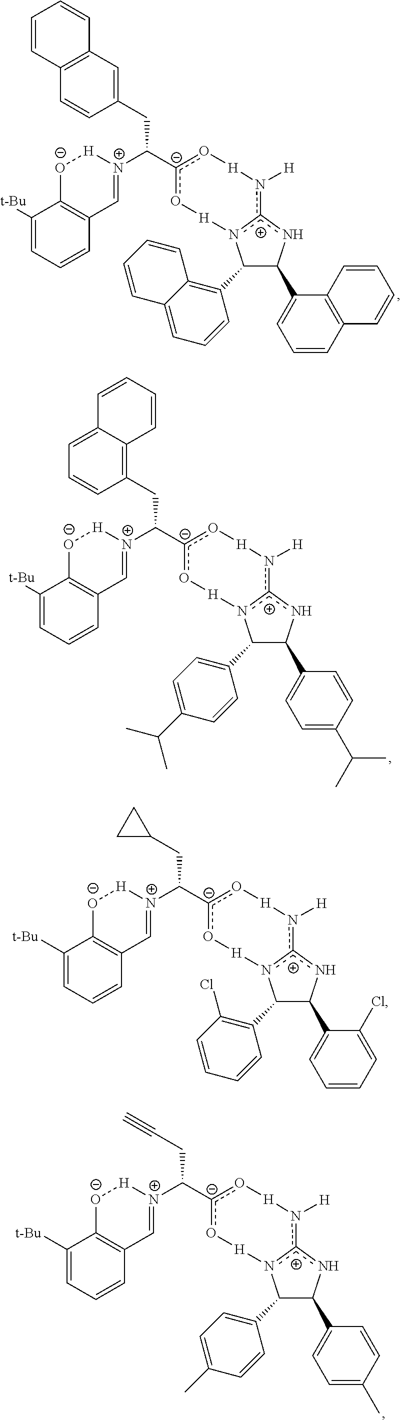 [chemical structure]
